SEQUENCE LISTING (9 sequences)

SEQ ID NO: 1 - protein, 488 aa, synthetic construct
  REGION 1..488: note = Synthetic: TFEB (wild type protein)
MASRIGLRMQLMREQAQQEEQRERMQQQAVMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVLKVPRARDPPVAT

SEQ ID NO: 2 - protein, 520 aa, synthetic construct
  REGION 1..520: note = Synthetic: (protein) sTFEB SS-TAT-TFEB
MLPGLALLLLAAWTARALNGYGRKKRRQRRRGMASRIGLRMQLMREQAQQEEQRERMQQQAVMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVLKVPRARDPPVAT

SEQ ID NO: 3 - protein, 490 aa, synthetic construct
  REGION 1..490: note = Synthetic: (protein) sTFEB-del30 (SS-TAT-TFEB-del 30)
MLPGLALLLLAAWTARALNGYGRKKRRQRRRGMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVLKVPRARDPPVAT

SEQ ID NO: 4 - protein, 467 aa, synthetic construct
  REGION 1..467: note = Synthetic: (protein) sTFEB-DD (SS-TAT-TFEB-DD)
MLPGLALLLLAAWTARALNGYGRKKRRQRRRGMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRADRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVLKVPRARDPPVAT

SEQ ID NO: 5 - DNA, 1563 bp, synthetic construct
  misc_feature 1..1563: note = Synthetic: DNA sTFEB (SS TAT_TFEB)
  source 1..1563: mol_type = other DNA
atgctgcccggtttggcactgctcctgctggccgcctggacggctcgggcgctgaacggttacggtcgtaagaagcgtcgtcagcgtcgtcgtggcatggcgtcacgcatagggttgcgcatgcagctcatgcgggagcaggcgcagcaggaggagcaacgggagcgcatgcagcaacaggctgtcatgcattacatgcagcagcagcagcagcagcaacagcagcagctcggagggccgcccaccccggccatcaatacccccgtccacttccagtcgccaccacctgtgcctggggaggtgttgaaggtgcagtcctacctggagaatcccacatcctaccatctgcagcagtcgcagcatcagaaggtgcgggagtacctgtccgagacctatgggaacaagtttgctgcccacatcagcccagcccagggctctccgaaacccccaccagccgcctccccaggggtgcgagctggacacgtgctgtcctcctccgctggcaacagtgctcccaatagccccatggccatgctgcacattggctccaaccctgagagggagttggatgatgtcattgacaacattatgcgtctggacgatgtccttggctacatcaatcctgaaatgcagatgcccaacacgctacccctgtccagcagccacctgaatgtgtacagcagcgacccccaggtcacagcctccctggtgggcgtcaccagcagctcctgccctgcggacctgacccagaagcgagagctcacagatgctgagagcagggccctggccaaggagcggcagaagaaagacaatcacaacttaattgaaaggagacgaaggttcaacatcaatgaccgcatcaaggagttgggaatgctgatccccaaggccaatgacctggacgtgcgctggaacaagggcaccatcctcaaggcctctgtggattacatccggaggatgcagaaggacctgcaaaagtccagggagctggagaaccactctcgccgcctggagatgaccaacaagcagctctggctccgtatccaggagctggagatgcaggctcgagtgcacggcctccctaccacctccccgtccggcatgaacatggctgagctggcccagcaggtggtgaagcaggagctgcctagcgaagagggcccaggggaggccctgatgctgggggctgaggtccctgaccctgagccactgccagctctgcccccgcaagccccgctgcccctgcccacccagccaccgtccccattccatcacctggacttcagccacagcctgagctttgggggcagggaggacgagggtcccccgggctaccccgaacccctggcgccggggcatggctccccattccccagcctgtccaagaaggatctggacctcatgctcctggacgactcactgctaccgctggcctctgatccacttctgtccaccatgtcccccgaggcctccaaggccagcagccgccggagcagcttcagcatggaggagggcgatgtgctgaaggtaccgcgggcccgggatccaccggtcgccacctaa

SEQ ID NO: 6 - DNA, 1473 bp, synthetic construct
  misc_feature 1..1473: note = Synthetic: DNA sTFEB-del30 (SS-TAT-TFEB-del30)
  source 1..1473: mol_type = other DNA
atgctgcccggtttggcactgctcctgctggccgcctggacggctcgggcgctgaacggttacggtcgtaagaagcgtcgtcagcgtcgtcgtggcatgcattacatgcagcagcagcagcagcagcaacagcagcagctcggagggccgcccaccccggccatcaatacccccgtccacttccagtcgccaccacctgtgcctggggaggtgttgaaggtgcagtcctacctggagaatcccacatcctaccatctgcagcagtcgcagcatcagaaggtgcgggagtacctgtccgagacctatgggaacaagtttgctgcccacatcagcccagcccagggctctccgaaacccccaccagccgcctccccaggggtgcgagctggacacgtgctgtcctcctccgctggcaacagtgctcccaatagccccatggccatgctgcacattggctccaaccctgagagggagttggatgatgtcattgacaacattatgcgtctggacgatgtccttggctacatcaatcctgaaatgcagatgcccaacacgctacccctgtccagcagccacctgaatgtgtacagcagcgacccccaggtcacagcctccctggtgggcgtcaccagcagctcctgccctgcggacctgacccagaagcgagagctcacagatgctgagagcagggccctggccaaggagcggcagaagaaagacaatcacaacttaattgaaaggagacgaaggttcaacatcaatgaccgcatcaaggagttgggaatgctgatccccaaggccaatgacctggacgtgcgctggaacaagggcaccatcctcaaggcctctgtggattacatccggaggatgcagaaggacctgcaaaagtccagggagctggagaaccactctcgccgcctggagatgaccaacaagcagctctggctccgtatccaggagctggagatgcaggctcgagtgcacggcctccctaccacctccccgtccggcatgaacatggctgagctggcccagcaggtggtgaagcaggagctgcctagcgaagagggcccaggggaggccctgatgctgggggctgaggtccctgaccctgagccactgccagctctgcccccgcaagccccgctgcccctgcccacccagccaccgtccccattccatcacctggacttcagccacagcctgagctttgggggcagggaggacgagggtcccccgggctaccccgaacccctggcgccggggcatggctccccattccccagcctgtccaagaaggatctggacctcatgctcctggacgactcactgctaccgctggcctctgatccacttctgtccaccatgtcccccgaggcctccaaggccagcagccgccggagcagcttcagcatggaggagggcgatgtgctgaaggtaccgcgggcccgggatccaccggtcgccacctaa

SEQ ID NO: 7 - DNA, 1404 bp, synthetic construct
  misc_feature 1..1404: note = Synthetic: DNA sTFEB-DD (SS-TAT-TFEB-DD)
  source 1..1404: mol_type = other DNA
atgctgcccggtttggcactgctcctgctggccgcctggacggctcgggcgctgaacggttacggtcgtaagaagcgtcgtcagcgtcgtcgtggcatgcattacatgcagcagcagcagcagcagcaacagcagcagctcggagggccgcccaccccggccatcaatacccccgtccacttccagtcgccaccacctgtgcctggggaggtgttgaaggtgcagtcctacctggagaatcccacatcctaccatctgcagcagtcgcagcatcagaaggtgcgggagtacctgtccgagacctatgggaacaagtttgctgcccacatcagcccagcccagggctctccgaaacccccaccagccgcctccccaggggtgcgagctggacacgtgctgtcctcctccgctggcaacagtgctcccaatagccccatggccatgctgcacattggctccaaccctgagagggagttggatgatgtcattgacaacattatgcgtctggacgatgtccttggctacatcaatcctgaaatgcagatgcccaacacgctacccctgtccagcagccacctgaatgtgtacagcagcgacccccaggtcacagcctccctggtgggcgtcaccagcagctcctgccctgcggacctgacccagaagcgagagctcacagatgctgagagcagggccgaccgcatcaaggagttgggaatgctgatccccaaggccaatgacctggacgtgcgctggaacaagggcaccatcctcaaggcctctgtggattacatccggaggatgcagaaggacctgcaaaagtccagggagctggagaaccactctcgccgcctggagatgaccaacaagcagctctggctccgtatccaggagctggagatgcaggctcgagtgcacggcctccctaccacctccccgtccggcatgaacatggctgagctggcccagcaggtggtgaagcaggagctgcctagcgaagagggcccaggggaggccctgatgctgggggctgaggtccctgaccctgagccactgccagctctgcccccgcaagccccgctgcccctgcccacccagccaccgtccccattccatcacctggacttcagccacagcctgagctttgggggcagggaggacgagggtcccccgggctaccccgaacccctggcgccggggcatggctccccattccccagcctgtccaagaaggatctggacctcatgctcctggacgactcactgctaccgctggcctctgatccacttctgtccaccatgtcccccgaggcctccaaggccagcagccgccggagcagcttcagcatggaggagggcgatgtgctgaaggtaccgcgggcccgggatccaccggtcgccacctaa

SEQ ID NO: 8 - protein, 18 aa, Homo sapiens
  REGION 1..18: note = misc_feature - human Amyloid Precursor Protein
MLPGLALLLLAAWTARAL

SEQ ID NO: 9 - protein, 13 aa, Human immunodeficiency virus
  REGION 1..13: note = misc_feature - TAT peptide from HIV
TVTVVRSVVSVVV

(19) United States
(12) Patent Application Publication
(10) Pub. No.: US 2025/0152741 A1
(43) Pub. Date: May 15, 2025

(54) NUCLEIC ACID CONSTRUCTS ENCODING A CELL PENETRATING PEPTIDE, SIGNAL SEQUENCE AND TRANSCRIPTION FACTOR EB AND USES THEREOF

(71) Applicant: LONDON HEALTH SCIENCES CENTRE RESEARCH INC., London (CA)

(72) Inventors: Stephen Pasternak, London (CA); Claudia Seah, Ilderton (CA); Adrianna Tsang, Toronto (CA); Jordan KRUPA, Calgary (CA)

(73) Assignee: LONDON HEALTH SCIENCES CENTRE RESEARCH INC., London, ON (CA)

(21) Appl. No.: 18/723,357

(22) PCT Filed: Dec. 1, 2022
(86) PCT No.: PCT/CA2022/051763

§ 371 Date: Jun. 21, 2024

Related U.S. Application Data
(60) Provisional application No. 63/293,394, filed on Dec. 23, 2021.

(51) Int. Cl. A61K 48/00; A61P 25/28; C07K 14/47; C12N 15/86

(52) U.S. Cl. CPC A61K 48/0058; A61P 25/28; C07K 14/4702; C12N 15/86; C12N 2710/10043

(57) ABSTRACT

A nucleic acid construct comprising a polynucleotide that encodes for a secretory protein, the secretory protein comprising a non-secretory protein having a signal sequence domain and a cell penetrating peptide (CPP) sequence domain. Also, methods of using the nucleic acid construct in the treatment of diseases.

### FIELD

The present disclosure relates generally to polypeptides, nucleic acid constructs and vectors for treating, ameliorating or preventing diseases.

### BACKGROUND

Currently there are no approved disease modifying treatments for most neurodegenerative diseases including Alzheimer's disease (AD) and Parkinson's disease (PD). Almost all neurodegenerative diseases are accompanied by the deposition of protein aggregates, possibly due to a failure to degrade these aggregates. For example, AD is characterized by the accumulation of pathological forms of beta-amyloid (Abeta) and tau along with a failure of the autophagy/lysosomal system for clearance [1-3]. Similarly, PD is characterized by the deposition of alpha synuclein (aSyn) in the brain, and failure of lysosomal clearance, including a massive loss of lysosomes [4]. Other authors have suggested that lysosomal failure may be common in other neurodegenerative diseases including Frontotemporal dementias [5].

Transcription Factor EB (TFEB) is a master regulator of autophagosome/lysosome function, driving hundreds of genes in the CLEAR (Coordinated Lysosomal Expression and Regulation) network [6-8]. TFEB normally remains inactive in the cytoplasm, bound to cytoplasmic proteins. When activated, TFEB is released and moves to the nucleus where it binds DNA to direct gene expression.

Patients and AD animal models show a reduction of active TFEB along with failure of lysosomal clearance. [1-3, 9] Conversely, increasing TFEB expression has been shown to reduce Abeta and tau deposition and improve cognitive function in mouse models. [9-12]. Similarly, overexpressed TFEB drives clearance of aSyn aggregates from cells lines [13], cultured dopaminergic/midbrain neurons overexpressing aSyn [14, 15] or cells treated with the dopaminergic toxin 6-hydroxydopamine [14]. TFEB overexpression protects neurons from neurodegeneration in a rat model or PD the effects of PD-causing toxins Rotenone [17, 18] and MPTP/MPP+ [4, 19]. Increasing TFEB function is therefore a promising strategy for the treatment of neurodegenerative disease.

Despite decades of work, gene transfer into the CNS remains a critical problem. The main solutions in use depend upon either on Cell Penetrating Peptides (CPPs) or viral transduction. CPPs such as the “TAT” peptide derived from the HIV virus allow macromolecules to cross cell membranes and the blood brain barrier. However, these methods require large amounts of protein to be delivered and have had few clinical successes to date [20]. Our own work has shown that while TAT-tagged peptides are rapidly transported into the brain, this same TAT peptide also allows materials to be rapidly cleared from the brain [21]. Viral transduction, on the other hand uses viruses to introduce a gene's DNA into individual cells and can be quite effective in cell and mouse models of disease. Many viral vectors including adenovirus, lentivirus, and adeno-associated virus (AAV) are under study. AAV is considered by many to be the vector of choice for therapy in the Central Nervous System because of reduced oncogenic potential (does not integrate into the genome), long expression times, safety in handling, relative freedom from immune reactions, and neurotropism (especially AAV9 variants) [22]. However, the core problem remains that viruses injected into solid tissue typically spread only a few millimeters. Similarly, systemically injected viruses are diluted and spread throughout the body are only able to transduce a fraction of the cells in a target organ. We still have no answer for diseases in which every single cell in a tissue needs to be transduced [20, 22, 23].

### SUMMARY

The present disclosure describes a system to improve spread of virally delivered polypeptides. In embodiments, the present disclosure comprises modifying a non-secretory protein (a protein that is normally present inside the cell only) such that it is to be secreted from virally transduced cells and taken up by surrounding cells. Unlike constructs of the prior art, the secretory polypeptide(s) presented in this disclosure is/are able to spread from cell to cell. In one embodiment the non-secretory protein is a transcription factor that is modified into a secretory transcription factor (sTF). In one embodiment, the sTF is a secreted TFEB (sTFEB) that can spread from cell to cell, increasing lysosome size and number, and reducing beta amyloid, tau and alpha-synuclein aggregates. sTFEB delivery using the system of the present disclosure in the brain is substantially more robust than conventional viral delivery, and also leads to increased lysosomal protein expression and reduced aggregated alpha-synuclein, aggregated tau, and beta amyloid. Nucleic acid construct(s) s that encode(s) for the secretory polypeptide(s) of the present disclosure can be included into vector system that have application in therapy, amelioration or prevention of a variety of diseases/conditions that affect the body. In embodiments, the disease/condition is associated with dysfunctional lysosomal clearance that can affect the skeleton, skin mucous membrane, heart, muscular tissues, peripheral nervous system, central nervous system, for example. The disease/condition that affect dysfunction lysosomal clearance include one or more, neurodegenerative diseases, including Alzheimer's disease, Parkinson's disease, and so forth and muscular diseases such as muscular dystrophy. The constructs of the present disclosure can also be used in the treatment, amelioration or prevention of infections caused by a pathogen. In addition, the construct of the present disclosure can be widely applicable to other neurodegenerative diseases and gene therapy paradigms.

In one embodiment, the present disclosure relates to a nucleic acid construct comprising a polynucleotide that encodes a secretory protein variant, the secretory protein variant comprising a non-secretory protein having a signal sequence domain and a cell penetrating peptide (CPP) sequence domain.

In one embodiment of the nucleic acid construct of the present disclosure, the non-secretory protein is a nuclear protein, a cytoplasmic protein or an organelle protein.

In another embodiment of the nucleic acid construct of the present disclosure, the non-secretory protein is a transcription factor.

In another embodiment of the nucleic acid construct of the present disclosure, the secretory protein variant is a secreted TFEB (sTFEB) comprising a wild-type TFEB having the signal sequence and the self-penetrating sequence.

In another embodiment of the nucleic acid construct of the present disclosure, the non-secretory protein is a HSP or Cox8.

In another embodiment of the nucleic acid construct of the present disclosure, the polynucleotide is devoid of a sequence that encodes a cytoskeletal binding site.

In another embodiment of the nucleic acid construct of the present disclosure, the polynucleotide is devoid of a sequence that encodes for a DNA binding domain.

In another embodiment of the nucleic acid construct of the present disclosure, the self-penetrating sequence includes a TAT sequence, Pep-1, PVEC, Polyarginine (R9, R8), Penetratin, PVEC, MPG, R6/W3, SAP, CyLoP-1, gH 625, GALA, TP10, CADY, L17E, MPPs, Ac-1, Ent ac-1, Ac-2, Peptide 3, RR5-App, RR4-App, RR3-aPP, TATp-D, R4-R4, R5, R5, [WR]4, Cyclic Tat, cFΦR4, Danamide D, Pro-(Xaa)4-Tyr, Cyclic sC18, RRRRΦF, BIM SAHB9 SAH-SOS1, 4-R, 4-W, Sp-CC-PEG2000, K10(QW)6, YTA4, v2 or W3.

In another embodiment of the nucleic acid construct of the present disclosure, the signal sequence domain is derived from of the human Amyloid Precursor protein

In another embodiment of the nucleic acid construct of the present disclosure, the construct comprises an inducible promoter or a constitutive promoter operatively linked to the polynucleotide that encodes for the non-secretory protein.

In another embodiment of the nucleic acid construct of the present disclosure, the promoter drives expression of the polynucleotide in the central nervous system.

In another embodiment of the nucleic acid construct of the present disclosure, the construct comprises a Glial Fibrillary Acidic Protein (GFAP) promoter, a CAG promoter, an SV40 promoter, a neuronal promoter or a EF1a promoter.

In another embodiment of the nucleic acid construct of the present disclosure, the polynucleotide comprises, consists essentially of, or consists of SEQ ID NO: 5.

In another embodiment of the nucleic acid construct of the present disclosure, the polynucleotide comprises, consists essentially of, or consists of SEQ ID NO: 6.

In another embodiment of the nucleic acid construct of the present disclosure, the polynucleotide comprises, consists essentially of, or consists of SEQ ID NO: 7.

In another embodiment, the present disclosure provides for a recombinant vector comprising a nucleic acid construct of the present disclosure.

In one embodiment, the recombinant vector is a viral vector.

In another embodiment, the viral vector is a retrovirus, lentivirus, adenovirus, adeno-associated virus and herpes simplex virus In another embodiment, the viral vector is an adeno associated virus (AAV).

In another embodiment, the present disclosure provides for a recombinant secretory protein variant comprising a non-secretory protein having a signal sequence domain and a cell penetrating peptide (CPP) domain.

In one embodiment of the recombinant secretory protein variant of the present disclosure, the non-secretory protein is a nuclear protein, a cytosolic protein or an organelle protein.

In another embodiment of the recombinant secretory protein variant of the present disclosure, the non-secretory protein is a transcription factor.

In another embodiment of the recombinant secretory protein variant of the present disclosure, the recombinant secretory protein is a recombinant secreted Transcription Factor EB (sTFEB) comprising a wild-type TFEB having the signal sequence domain and the self-penetrating peptide domain.

In another embodiment of the recombinant secretory protein variant of the present disclosure, the recombinant secretory protein is a recombinant secreted HSP or a recombinant secreted Cox8.

In another embodiment of the recombinant secretory protein variant of the present disclosure, the CPP domain includes a TAT sequence, Pep-1, PVEC, Polyarginine (R9, R8), Penetratin, PVEC, MPG, R6/W3, SAP, CyLoP-1, gH 625, GALA, TP10, CADY, L17E, MPPs, Ac-1, Ent ac-1, Ac-2, Peptide 3, RR5-App, RR4-App, RR3-aPP, TATp-D, R4-R4, R5, R5, [WR]4, Cyclic Tat, cFΦR4, Danamide D, Pro-(Xaa)4-Tyr, Cyclic sC18, RRRRΦF, BIM SAHB9 SAH-SOS1, 4-R, 4-W, Sp-CC-PEG2000, K10(QW)6, YTA4, v2 or W3.

In another embodiment of the recombinant secretory protein variant of the present disclosure, the signal sequence domain includes a signal peptide of the human Amyloid Precursor protein.

In another embodiment of the recombinant secretory protein variant of the present disclosure, the recombinant secretory protein is devoid of a cytoskeletal binding site (sTFEB del30).

In another embodiment of the recombinant secretory protein variant of the present disclosure, the recombinant secretory protein is devoid of a DNA binding domain (sTFEB-DD).

In another embodiment of the recombinant secretory protein variant of the present disclosure, the recombinant secretory protein comprises, consists essentially of, or consists of SEQ ID NO: 2.

In another embodiment of the recombinant secretory protein variant of the present disclosure, the recombinant secretory protein comprises, consists essentially of, or consists of SEQ ID NO: 3.

In another embodiment of the recombinant secretory protein variant of the present disclosure, the recombinant secretory protein comprises, consists essentially of, or consists of SEQ ID NO: 4.

In another embodiment, the present disclosure relates to a method of treating, ameliorating or preventing a disease in a subject, the method comprising administering to the subject a vector, wherein the vector comprises a polynucleotide construct that encodes a recombinant secretory protein variant comprising a non-secretory protein having a signal sequence domain and a cell penetrating peptide (CPP) domain.

In one embodiment of the method, the vector is a viral vector.

In another embodiment of the method, the viral vector is a retrovirus, lentivirus, adenovirus, adeno-associated virus and herpes simplex virus

In another embodiment of the method, the viral vector is AAV.

In another embodiment of the method, the non-secretory protein is a nuclear protein, an organelle protein or a cytoplasmic protein.

In another embodiment of the method, the non-secretory protein is a transcription factor.

In another embodiment of the method, the transcription factor is Transcription Factor EB (TFEB), and wherein the recombinant secretory protein variant comprises a wild-type TFEB having the signal sequence domain and the cell-penetrating peptide domain.

In another embodiment of the method, the non-secretory protein is a HSP or Cox8.

In another embodiment of the method, the CPP domain includes a TAT sequence, Pep-1, PVEC, Polyarginine (R9, R8), Penetratin, PVEC, MPG, R6/W3, SAP, CyLoP-1, gH 625, GALA, TP10, CADY, L17E, MPPs, Ac-1, Ent ac-1, Ac-2, Peptide 3, RR5-App, RR4-App, RR3-aPP, TATp-D, R4-R4, R5, R5, [WR]4, Cyclic Tat, cFΦR4, Danamide D, Pro-(Xaa)4-Tyr, Cyclic sC18, RRRRΦF, BIM SAHB9 SAH-SOS1, 4-R, 4-W, Sp-CC-PEG2000, K10(QW)6, YTA4, v2 or W3.

In another embodiment of the method, the signal sequence domain includes a signal peptide of the human Amyloid Precursor protein.

In another embodiment of the method, the recombinant secretory protein variant is devoid of a cytoskeletal binding site (sTFEB del30).

In another embodiment of the method, the recombinant secretory protein comprises, consists essentially of, or consists of SEQ ID NO: 2.

In another embodiment of the method, the recombinant secretory protein comprises, consists essentially of, or consists of SEQ ID NO: 3.

In another embodiment of the method, the recombinant secretory protein comprises, consists essentially of, or consists of SEQ ID NO: 4.

In another embodiment of the method, the disease is associated with dysfunctional lysosomal clearance.

In another embodiment of the method, the disease affects the skeleton, skin, mucous membrane, heart, liver, hematopoietic system, musculature, peripheral nervous system and central nervous system.

In another embodiment of the method, the disease is a neurodegenerative disease.

In another embodiment of the method, the neurodegenerative disease is Alzheimer's disease or Parkinson's disease.

In another embodiment of the method, the disease is caused by a pathogen.

In another embodiment of the method, the pathogen is a virus, a bacterium or a fungus.

In another embodiment of the method, the disease is muscular dystrophy or MS.

In another embodiment, the present disclosure relates to a use of a vector as defined in the present disclosure in the treatment of a disease.

In one embodiment of the use, the disease is associated with dysfunctional lysosomal clearance.

In another embodiment of the use, the disease affects the skeleton, skin, mucous membrane, heart, liver, hematopoietic system, musculature, peripheral nervous system and central nervous system.

In another embodiment of the use, the disease is a neurodegenerative disease.

In another embodiment of the use, the neurodegenerative disease is Alzheimer's disease or Parkinson's disease.

In another embodiment of the use, the disease is caused by a pathogen.

In another embodiment of the use, the pathogen is a virus, a bacterium or a fungus.

### BRIEF DESCRIPTION OF THE DRAWINGS

The following figures illustrate various aspects and preferred and alternative embodiments of this disclosure.

FIG. 1. Figure illustrating an overview of constructs of the present disclosure. Vectors are designed to express sTFEB (secreted TFEB). In these embodiments the vectors are designed to express sTFEB-fused to Green Fluorescent Protein (GFP), where in this system GFP is used to easily follow the spread of sTFEB using a fluorescence microscope. In some embodiments, non-secreted Red Fluorescent Protein (RFP) to distinguish transduced cells from non-transduced cells that have taken up sTFEB (when following the spread or making a distinction between transduced and non-transduced is not necessary, the fluorescent proteins may be removed to save space in the vector, improve expression and remove a potential target of the immune system). In embodiments, the construct comprises a secretory signal sequence and a Cell Penetrating Peptide (CPP; in this instance the TAT peptide from HIV). In embodiments, the sTFEB lacks a cytoskeletal binding site (CC; sTFEB del30). In embodiments, these vectors contain luciferase for live animal imaging in place of GFP. In embodiments, the signal sequence (in this example is the signal peptide of the human Amyloid Precursor protein) ‘SS’ directs the TFEB into the secretory system. Once secreted, the ‘CPP’ domain allows the secreted protein to be taken up from the extracellular space by untransduced cells. The ‘CC’ domain of TFEB binds cytoskeletal structures; when this is deleted, TFEB translocates constitutively to the nucleus to drive gene expression (and is therefore always active). The HLH domain is required for DNA binding; deleting this domain inactivates TFEB mediated gene activation, and is used as a control for proof of principle studies.

FIG. 2. Microphotographs showing that sTFEB-GFP spreads from cell to cell in Neuroblastoma cells. Microphotographs of N2A cells transfected with sTFEB-GFP (top), STFEB-del30-GFP (middle) and sTFEB-DD-GFP (bottom). Cells are immunostained with LAMP1 (magenta). The rightmost panel shows a colocalization channel, comparing the overlap of STFEB-GFP and lysosomes (LAMP1) by Imaris software (Bitplane). Cells that are transfected with sTFEB-GFP also express RFP and are marked by arrowheads. All of the other cells that are green have taken up GFP from the media. Cells that are transfected with sTFEB-GFP show punctate cytoplasmic and nuclear staining, depending on how much sTFEB-GFP is taken up. Some STFEB-GFP in the cytoplasm colocalizes with lysosomes. Deleting the cytoskeletal binding domain (sTFEB-del30) increases the number of cells with nuclear GFP signal and reduces the amount of sTFEB on lysosomes. Deleting both DNA binding and cytoskeletal binding domains (STFEB-DD) leads to cytoplasmic GFP expression, but also less lysosomal labeling.

FIG. 3. sTFEB is delivered from cell to cell with high efficiency. Microphotographs showing that sTFEB (green) but not adenovirus (red) spreads from cell to cell. Cells in the left panel are transduced with adenovirus encoding sTFEB-GFP and a non-secreted Red Fluorescent Protein. Media that had been conditioned by these cells, is then added to a second dish of cells (right panels-neuroblastoma cells and cultured human iPSC-derived human neurons.) which then become green (but not red) demonstrating that they have taken up the sTFEB-GFP, but not the virus particles themselves.

FIGS. 4A to 4C. sTFEB increases the expression of Cathpsin-B, lysosomes size and lysosomal number. 4A) Microphotographs of N2a cells (4A-top panels) or iPSC-derived neurons (4A-bottom panels) were allowed to take up sTFEB del30 or TFEB/inactive sTFEB-DD-GFP for 48 hours from conditioned media. Magic Red (Immunochemistry Technologies) fluorescent substrate for Cathepsin B was added to the media and cells were imaged live. 4B) Lysosome size and number were quantitated using Imaris software. N2A cells which had taken up the constitutively active sTFEB-del30 (compared to control cells) had significantly more lysosomes, larger lysosomes and more CatB enzyme activity (generated by integrating intensity*area). 4C) Lysosome size and number were quantitated using Imaris software in human iPSC-derived neurons. Neurons which had taken up the constitutively active sTFEB-del30 (compared to control cells) had significantly more lysosomes, larger lysosomes and more CatB enzyme activity (generated by integrating intensity*area).

FIGS. 5A-5B. sTFEB increases the levels of transcription of lysosomal genes (5A) and lysosomal proteins (5B). 5A) N2A cells were treated with constitutively active TFEB-del30 or inactive sTFEB-DD and gene expression of expression was analyzed by rtPCR and normalized to cells exposed to normal media. sTFEB increases Lysosomal gene expression. 5B) N2A cells were treated with sTFEB, constitutively active TFEB-del30 or inactive sTFEB-DD and cells were analyzed by Western Blotting. sTFEB increases expression of LAMP1 and CatD proteins but not tubulin (control).

FIGS. 6A-6B. Constitutively active sTFEB-del30 decreases beta-Amyloid in cells. N2A neuroblastoma cells were transfected with either the inactive TFEB-DD-GFP (FIG. 6A) or constitutively active sTFEB-del30 (FIG. 6B). After 48 hours, they were loaded overnight with 500 nM of fluorescently labeled beta-amyloid-42 (shown in red). After a further 48 hours of incubation, cells were fixed and imaged. Photomicrographs illustrate that cells that contain inactive TFEB-DD-GFP demonstrated much brighter red signal from beta-amyloid than cells which had taken up constitutively active TEFB-GFP.

FIGS. 7A-7B. Constitutively active sTFEB-del30 reduces neurofibrillary tangles. Human iPSC-derived neurons were transduced with mutant Tau (Tau P301L)-blue fluorescent protein (shown in the photomicrographs in cyan) and treated with tau pre-formed fibrils to generate neurofibrillary tangles. Cells were then exposed to media containing inactive TFEB-DD-GFP (FIG. 7A) or constitutively active sTFEB-del30 (FIG. 7B). Photomicrographs show that cells exposed to inactive TFEB were filled with Tau filaments (FIG. 7A), while cells exposed to active TFEB-del30 have almost no recognizable filaments (FIG. 7B).

FIGS. 8A-8D. Constitutively active sTFEB reduces alpha-synuclein deposits. SHSY5Y neuroblastoma cells (8A and 8B) and human iPSC-derived Neurons (8C and 8D) were transduced with aSyn A53T-BFP (green) and either TFEB-DD-GFP (inactive, 8A and 8C) or sTFEB-del30-GFP (constitutively active, 8B and 8D). After 5 days cells were fixed and imaged by confocal microscopy. The photomicrographs show that cells with which had taken up inactive sTFEB-DD-GFP had cytoplasmic synuclein inclusions (arrowheads, 8A and 8C); cells which took up active STFEB-del30-GFP did not (8B and 8D).

FIG. 9. sTFEB-del30-GFP greatly increases TFEB expression in the brain compared to wild-type TFEB-GFP, increases lysosome staining and reduces alpha-synuclein. A 14-month-old homozygous M83 mouse received a stereotaxic intraventricular injection of 10\hat{ }7 infectious units of adenovirus overexpressing constitutively active sTFEB-del30-GFP on the left and a conventional wt-TFEB-GFP on the right. After 7 days, mouse brain was perfused, fixed, cryosectioned, and immunostained with an antibody against GFP (green), LAMP1 (red), and aggregated phosphorylated-synuclein, and imaged on a Leica TCS SP8 confocal microscope. Top row photomicrographs showing that TFEB-GFP expression is much higher on the side received the sTFEB construct. Middle Row The side injected with constitutively active TFEB shows increased LAMP1 staining overall (increased lysosomes), and on high magnification, show increased numbers of lysosomes. Bottom Row: Tissue was stained with an antibody against phosphorylated alpha-synuclein, which is specific to pathological synuclein deposits, and this is reduced on the side injected with sTFEB.

FIG. 10A-10B. 10A: Photomicrograph of mouse brain showing that sTFEB-del30 clears beta-amyloid. A 12-month-old 3×TG Alzheimer's disease mouse was injected into the hippocampus with an adenovirus expressing TFEB-del30-GFP on the left and an adenovirus expressing only a wt-GFP on the right. After 1 week, the mouse was sacrificed and stained for GFP (green) and Beta Amyloid (yellow). The left side injected with sTFEB-del30-GFP shows greatly increased expression and spread compared to the expression of non-spreading GFP. 10B: Amyloid plaques were counted in each hippocampus and the cortex above the hippocampus and the results are shown in the graph of FIG. 10B. FIGS. 10A and 10B show that are fewer plaques on the hippocampus and cortex sTFEB-del30 side (left side of 10A, sTFEB) compared to the control side (right side of 10A, GFP).

FIGS. 11A-11D. sTFEB spreads beyond its injection sites to clear beta-amyloid. 11A: staining for GFP at level of injection. 11B Staining for GFP 3 mm anterior to the injection site. 11C: Amyloid-42 immunostaining at the level of injection. 11D: enlargement of small rectangles of FIG. 11C.

FIG. 12. Alignment of SEQ ID NO: 2 and SEQ ID NO: 5.

### DETAILED DESCRIPTION

### Abbreviations

Unless defined otherwise, all technical and scientific terms used herein have the same meaning as commonly understood by one of ordinary skill in the art to which this disclosure belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this disclosure.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

The term “administering” includes any method of delivery of a construct, vector or composition of the present disclosure, including a pharmaceutical composition, vaccine or therapeutic agent, into a subject's system or to a particular region in or on a subject. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “amino acid” is known in the art. In general the abbreviations used herein for designating the amino acids and the protective groups are based on recommendations of the IUPAC-IUB Commission on Biochemical Nomenclature (see Biochemistry (1972) 11:1726-1732). For the amino acids relevant to the present disclosure the designations are: M: methionine, R: arginine, G: glycine, E: glutamic acid, L: leucine, F: phenylalanine. In certain embodiments, the amino acids used in the application of this disclosure are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

As used herein, the term “fragment” refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a full-length peptide, polypeptide or protein. In one embodiment, a fragment of a full-length protein retains activity of the full-length protein, e.g., immunogenic activity. In another embodiment, the fragment of the full-length protein does not retain the activity of the full-length protein, e.g., non-immunogenic activity.

As used herein, the terms “subject” or “patient” are used interchangeably. As used herein, the terms “subject” and “subjects” refers to either a human or non-human animal.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably to refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “recombinant” polynucleotide means a polynucleotide of genomic, cDNA, semi-synthetic, or synthetic origin, which either does not occur in nature or is linked to another polynucleotide in a non-natural arrangement. An “oligonucleotide” refers to a single stranded polynucleotide having less than about 100 nucleotides, less than about, e.g., 75, 50, 25, or 10 nucleotides.

The terms “polypeptide”, “peptide” and “protein” (if single chain) are used interchangeably herein to refer to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

In certain embodiments, polypeptides of the disclosure may be synthesized chemically, ribosomally in a cell free system, or ribosomally within a cell. Chemical synthesis of polypeptides of the disclosure may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full-length ligation product having a native peptide bond at the ligation site. Full length ligation products are chemically identical to proteins produced by cell free synthesis. Full length ligation products may be refolded and/or oxidized, as allowed, to form native disulfide-containing protein molecules (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91:12544-12548; M. Schnlzer, et al., Science (1992): vol., 3256, p 221; and K. Akaji, et al., Chem. Pharm. Bull. (Tokyo) (1985) 33:184).

“Recombinant expression vector” refers to a genetically-modified polynucleotide construct that permits the expression of an mRNA, protein, polypeptide, peptide or fragment thereof by a host cell, when the vector is contacted with the host cell under conditions suitable to have the mRNA, protein, polypeptide, peptide or fragment thereof expressed within the host cell. The vectors of the disclosure are not naturally-occurring as a whole.

### Overview

Presented herein are novel secreted/diffusible protein variants or versions designed to spread out from the sites of a transduction injection and be taken up by surrounding cells to increase its effective tissue distribution. To this end, nucleotide constructs have been designed to express the novel protein variants of the present disclosure.

In one embodiment, the present disclosure provides for a secretory variant of a non-secretory protein that improves its spread through a tissue when delivered by a vector, such as a viral vector.

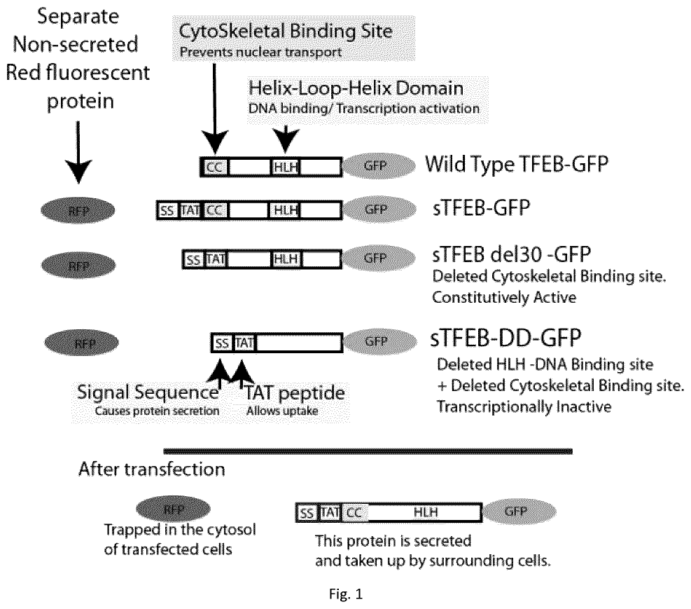

In one embodiment, the secretory variant protein includes at its N-terminus a signal sequence and a cell penetrating peptide (CPP), such as the TAT peptide derived from the transcription factor of HIV, which allows uptake of the resulting variant secretory protein across cell membranes. Signal peptides at the amino-terminal region of the variant secretory proteins target the protein to the ER and Golgi network (the secretory pathway) for the synthesis and secretion of the variant protein. The signal sequence and the CPP sequence may be coupled to the N-terminus of the secreted/diffusible protein as illustrated in FIG. 1.

The signal sequence may be any suitable signal sequence that directs the protein to the ER and Golgi network. Examples include the signal peptide of the human Amyloid Precursor protein.

Non-limiting examples of CPP that may be suitable for the constructs of the present disclosure include, Pep-1, PVEC, Polyarginine (R9, R8), Penetratin, PVEC, MPG, R6/W3, SAP, CyLoP-1, gH 625, GALA, TP10, CADY, L17E, MPPs, Ac-1, Ent ac-1, Ac-2, Peptide 3, RR5-App, RR4-App, RR3-aPP, TATp-D, R4-R4, R5, R5, [WR]4, Cyclic Tat, cFΦR4, Danamide D, Pro-(Xaa)4-Tyr, Cyclic sC18, RRRRΦF, BIM SAHB9 SAH-SOS1, 4-R, 4-W, Sp-CC-PEG2000, K10(QW)6, YTA4, v2 or W3 (see Daniela Kalafatovic and Ernest Giralt, Molecules. 2017 November; 22 (11): 1929).

The non-secretory proteins that can be made into the secretory variants of the present disclosure include, without limitation, nuclear, cytoplasmic and organelle non-secretory proteins such as TFEB, a stress response protein (e.g., heat shock protein HSP) or mitochondria protein (e.g., Cox8) and so forth.

In one embodiment, the non-secretory protein to be modified into a secretory variant is a transcription factor and in embodiments, the secretory variant is a secretory transcription factor (sTF). In one embodiment, the sTF is a secretory/diffusible TFEB (sTFEB). In one embodiment of the novel sTFEB variant of the present disclosure, wild-type TFEB (SEQ ID NO: 1) includes at its N-terminus a Signal Sequence (which causes secretion of the resulting protein) and a Cell Penetrating Peptide (CPP; in this case the TAT peptide from HIV), which allows uptake of the resulting variant protein across cell membranes (see FIG. 1, sTFEB, SEQ ID NO:2). Signal peptides at the amino-terminal region of the secretory proteins target the protein to the ER and Golgi network (the secretory pathway) for the synthesis and secretion of the protein. The signal sequence and the CPP sequence may be coupled to the N-terminus of the TFEB as illustrated in FIG. 1.

In one embodiment, the secretory protein variants of the present disclosure are constitutively active by deleting their cytoskeletal binding site (when present). So, using sTFEB as an example, by removing the cytoskeletal binding site that the resulting modified secretory protein variant is transported to the nucleus by default [25]. In another embodiment, the secretory protein variant is further modified by deleting a DNA binding site (for example when the secretory protein variant is a transcription factor) such that the secretory protein variant of this embodiment cannot activate gene expression.

In embodiments the present disclosure provides for new versions of sTFEB that are constitutively active (sTFEB-Delta30; SEQ ID NO:3) by deleting wild-type TFEB's cytoskeletal binding site, so the resulting protein is transported to the nucleus by default [25]. An inactive form of sTFEB (STFEB-DD; SEQ ID NO:4) was generated by deleting both the cytoskeletal and the DNA binding sites [26] so the TFEB protein cannot bind DNA to activate gene expression.

In embodiments, the polynucleotide constructs of the present disclosure that encode for the novel secreted/diffusible transcription factors can be inserted into a variety of suitable vectors, including expression plasmids, or viruses including Adenovirus and Adeno Associated Virus 9 (AAV9) retrovirus, lentivirus and herpes simplex virus. As such, in embodiments, the present disclosure relates also to vectors carrying the nucleotide constructs of the present disclosure. Viral constructs used in this example is a minimal EF1a promoter to drive expression in the Central Nervous System, but other cell-type specific promoters could be used. Non-limiting examples of promoters that can be used include Glial Fibrillary Acidic Protein (GFAP) promoter, a CAG promoter, an SV40 promoter, a neuronal promoter or an EF1a promoter.

In embodiments, a set of constructs have been designed with sTFEB in Adeno Associated Virus 5 (AAV5) driven from the Glial Fibrillary Acidic Protein (GFAP) promoter for preferential expression in glia/astrocytes [27]. These GFAP constructs can reduce the metabolic load of sTFEB production on neurons. Adenovirus is used in most preliminary experiments because AAV requires weeks for expression, making cell culture experiments hard, and is too small to include RFP to identify transduced cells.

In one embodiment, a sTFEB of the present disclosure comprises, consists essentially of, or consists of the amino acid sequence of SEQ ID NO: 2.

In another embodiment, a sTFEB of the present disclosure comprises, consists essentially of, or consists of the amino acid sequence of SEQ ID NO: 3.

In another embodiment, a sTFEB of the present disclosure comprises, consists essentially of, or consists of the amino acid sequence of SEQ ID NO: 4.

In one embodiment, a construct of the present disclosure comprises, consists essentially of, or consists of the nucleotide sequence that encodes sTFEB (SEQ ID NO: 5).

In another embodiment, a construct of the present disclosure comprises, consists essentially of, or consists of the nucleotide sequence that encodes sTFEBdel30 (SEQ ID NO: 6).

In another embodiment, a construct of the present disclosure comprises, consists essentially of, or consists of the nucleotide sequence that encodes sTFEB-DD (SEQ ID NO: 7).

In another embodiment, a construct of the present disclosure comprises, consists essentially of, or consists of the amino acid sequence of SEQ ID NO: 1, or SEQ ID NO:2, or SEQ ID NO:3 or SEQ ID NO:4 attached to a Green Fluorescent Protein ta (GFP) or a small peptide tag to aid in detection (e.g., an HA tag) or any other protein that assist in detection.

The secretory protein variant versions of the present disclosure can be isolated or purified from cells separately in culture, or from a recombinant source. For instance, a DNA fragment encoding a polypeptide construct of the present disclosure can be subcloned into an appropriate vector using well-known molecular genetic techniques. The fragment can be transcribed and the polypeptide subsequently translated in vitro. Commercially available kits also can be employed. The polymerase chain reaction optionally can be employed in the manipulation of nucleic acids.

In embodiments, the present disclosure provides a vector comprising the nucleic acid construct of the present disclosure. The nucleic acid construct of the present disclosure can be inserted into a suitable vector. The selection of vectors and methods to construct them are commonly known in the art and are described in general technical references.

Suitable vectors include those designed for propagation and expansion or for expression or both. Examples of suitable vectors include, for instance, plasmids, plasmidliposome complexes, CELid vectors and viral vectors, e.g., parvoviral-based vectors (i.e., AAV vectors), retroviral vectors, herpes simplex virus (HSV)-based vectors, adenovirus-based vectors, lentivirus, and poxvirus vectors. Any of these expression constructs can be prepared using standard recombinant DNA techniques.

In one embodiment, the vector is an AAV vector including suitable serotypes of AAV such as AAV1, AAV2, AAV3, AAV4, AAV5, AAV6, AAV7, AAV8, AAV9, and variants thereof.

The AAV vector may be packaged in a capsid protein, or fragment thereof, of any of the AAV serotypes described herein.

In an embodiment of this disclosure, the vector is a recombinant expression vector.

In embodiments, the vector of the present disclosure comprises regulatory sequences that permit one or more of the transcription, translation, and expression of nucleic acid comprised in the vector in a cell transfected with the vector or infected with a virus that comprises the vector. As used herein, “operably linked” sequences include both regulatory sequences that are contiguous with the nucleotide sequence encoding the sTFEB of the present disclosure and regulatory sequences that act in trans or at a distance to control the nucleotide sequence encoding the sTFEB.

The regulatory sequences may include appropriate transcription initiation, termination, promoter and enhancer sequences; RNA processing signals such as splicing and polyadenylation (polyA) signal sequences; sequences that stabilize cytoplasmic mRNA; sequences that enhance translation efficiency (i.e., Kozak consensus sequence); sequences that enhance protein stability; and when desired, sequences that enhance secretion of the encoded product. PolyA signal sequences may be synthetic or may be derived from many suitable species, including, for example, SV-40, human and bovine.

When the vector is for administration to a host (e.g., human), the vector (e.g., AAV) is preferably dead (i.e., no replication, unable to replicate) or is attenuated or it has a low replicative efficiency in a target cell (e.g., no more than about 1 progeny per cell or, more preferably, no more than 0.1 progeny per cell are produced). Replication efficiency can readily be determined empirically by determining the virus titer after infection of the target cell.

Other therapeutic proteins that can be used as the non-secretory protein of the nucleic acid constructs of the present disclosure include heat shock proteins like HSP70 to aid in cell stress, and the mitochondria cytochrome c oxidase subunit 8 (Cox8) to target mitochondria in the cell.

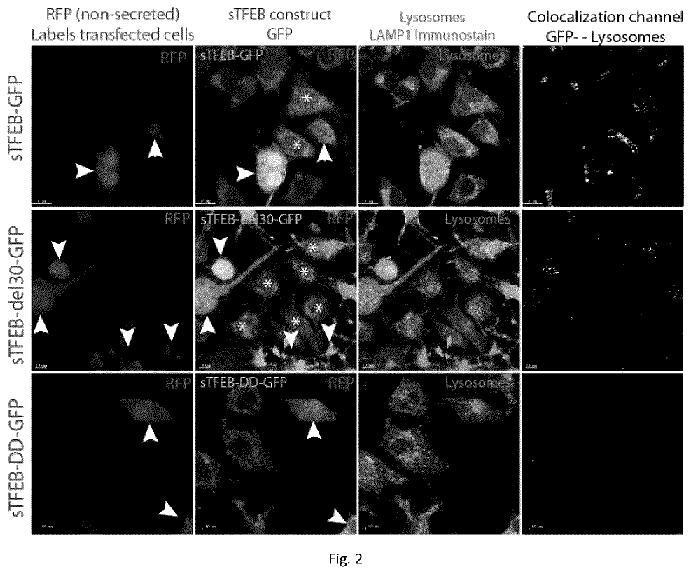

Without being limited to any mechanism of action, FIG. 2 illustrates how constructs of the present disclosure that encode for sTFEB function in cultured neuroblastoma cells (N2A cells). In this experiment, DNA is introduced into plasmids, and only a fraction of the cells actually take up the DNA. Cells that have taken up plasmid DNA are green from the TFEB construct having green fluorescent protein (GFP) and red because of the non-secreted RFP signal and indicated by arrowheads. All of the other green cells (that do not include red staining) have taken up sTFEB-GFP from the media. With full length sTFEB-GFP, cells have punctate green signal in cytoplasm, and variable nuclear staining. We have stained the lysosomes with an antibody to LAMP1. Using Imaris software, it is possible to select the brightest GFP and LAMP1 pixels, to generate a white colocalization channel, showing sTFEB is partly colocalized with LAMP1-labled lysosomes. Deleting the cytoplasmic retention domain (del30-deletion) makes the construct constitutively active and increases the number of cells that have sTFEB-del30-GFP in their nuclei, and reduces the amount of sTFEB-del30-GFP that colocalized with lysosomes. sTFEB DD, with both the cytoskeletal and the DNA binding domains deleted, is primarily cytoplasmic with reduced colocalization with lysosomes.

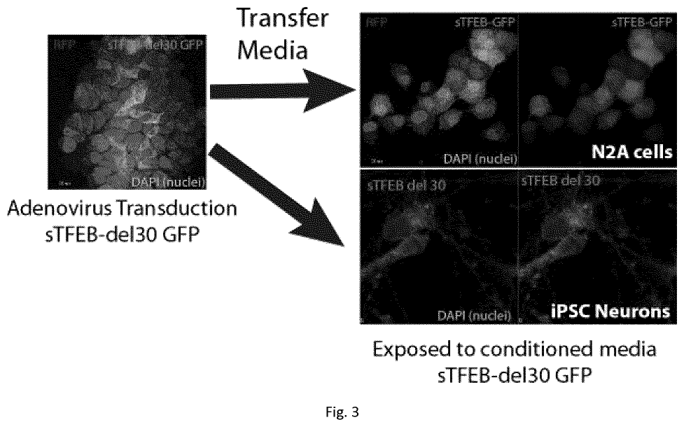

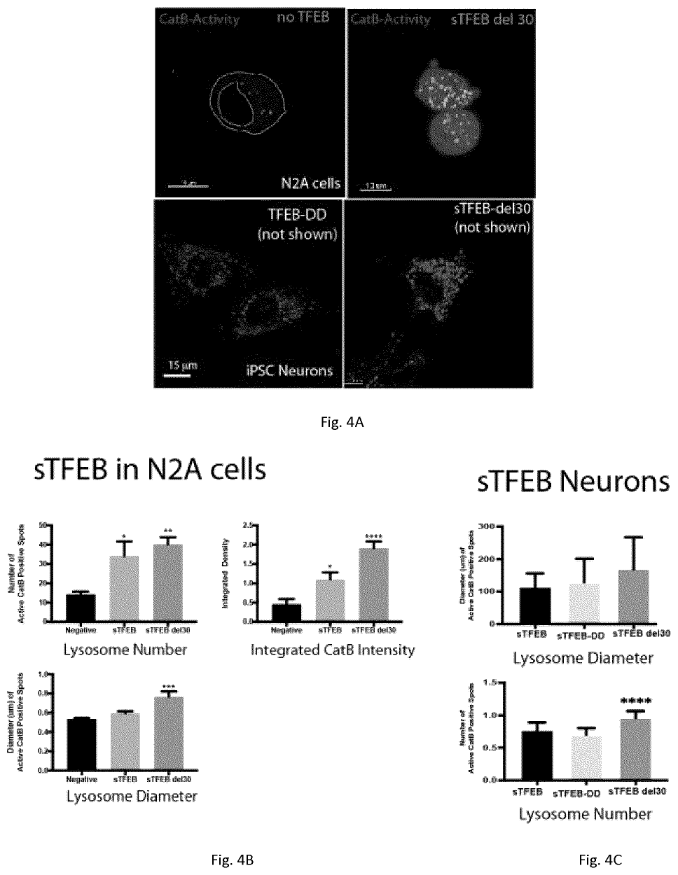

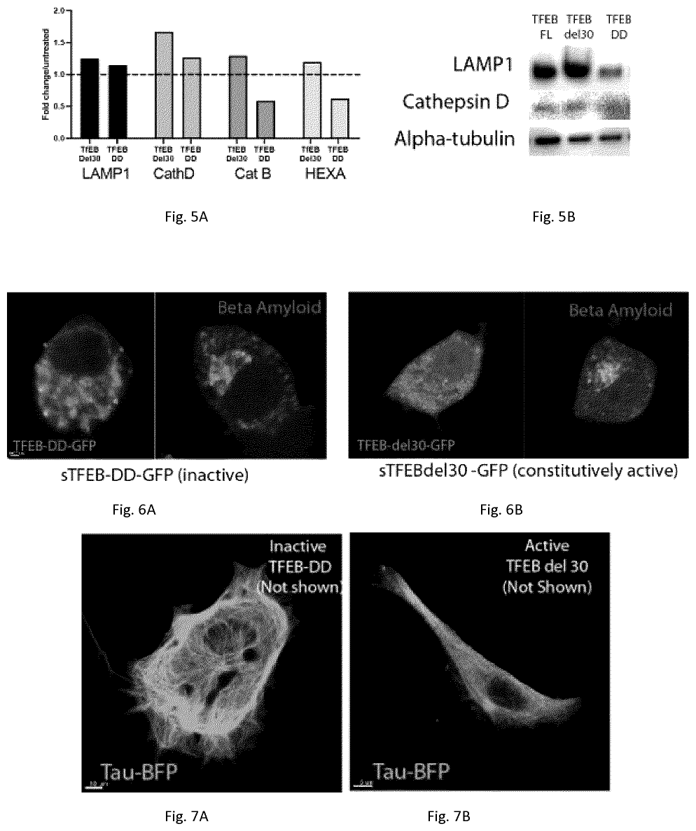

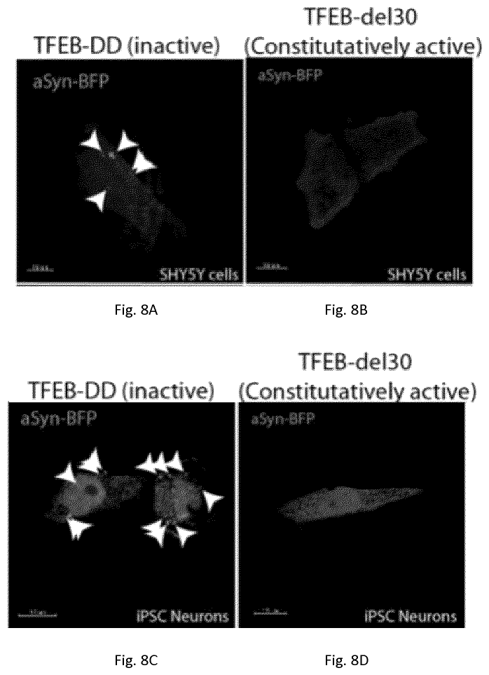

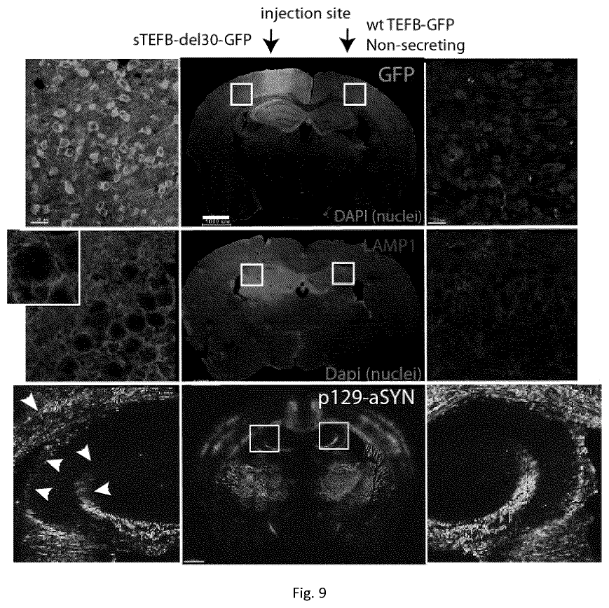

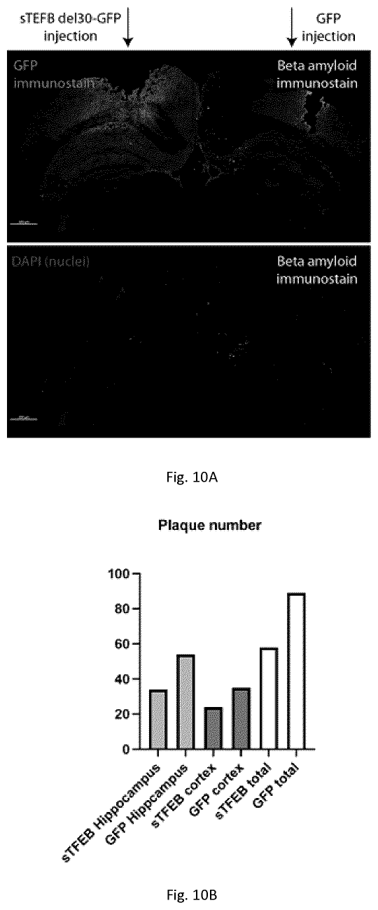

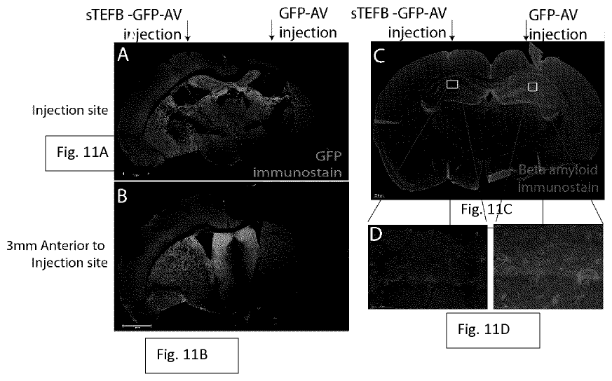

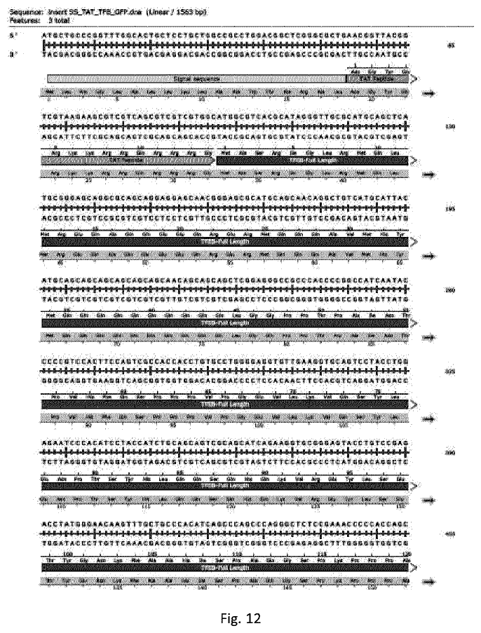

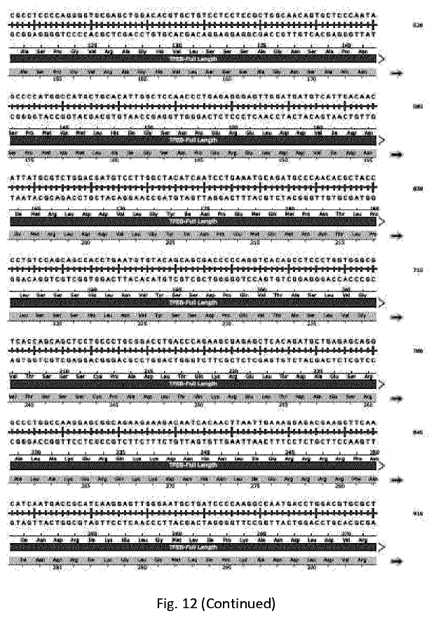

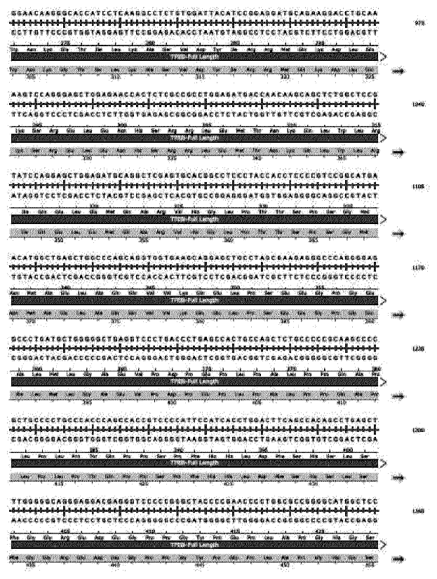

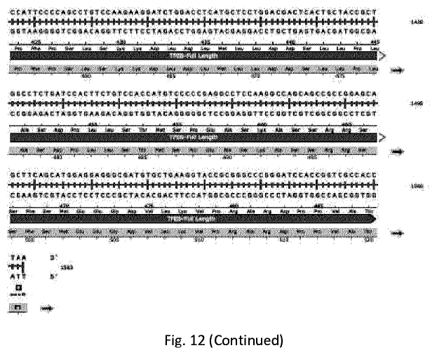

The vectors of the present disclosure achieve nearly 100% transduction efficiency in cell lines and human iPSC-derived neurons (FIG. 3). When media is taken from a dish of sTFEB-transduced cells, it is able to transfer sTFEB into nearly 100% of recipient cells including human neurons generated from inducible programmable stem cell (iPSC-derived neurons). sTFEB-del30 is present in most nuclei of recipient cells. Uptake of sTFEB from the media (particularly constitutively active sTFEBdel30) increases lysosomal size and number (see FIG. 4) and transcription/translation of lysosomal genes (FIG. 5). Uptake of sTFEB-del30 also leads to the clearance of beta amyloid (FIG. 6), neurofibrillary tangles (FIG. 7) and alpha-synuclein inclusions. Stereotactic injections of a vector of the present disclosure into Alzheimer's disease triple transgenic (APP/PSI/Tau) and Parkinson's disease (M83) mice demonstrate that this vector appears to result in the dramatic spreading of sTFEB through the brain (compared to wild type non-secreted TFEB; FIG. 9 and FIGS. 11A-11C), increasing expression of the lysosomal protein LAMP1 and reducing Alpha synuclein (FIG. 9) and beta-Amyloid plaques (FIGS. 10A-10B, 11C-11D). The mouse of FIGS. 10A-10B was treated for only 1 week, yet plaques were reduced.

The constructs of the present disclosure provide for a new method of gene delivery which overcomes the one of the leading problems of gene therapy. Currently there are no widely accepted disease modifying therapies for muscular and neurodegenerative diseases. The present disclosure provides for new constructs that express secretory protein variants, such as secretory transcription factor variants that were shown to increase lysosomal clearance in human neurons and mouse brains. The system of the present disclosure can transfer an engineered form of TFEB from virally transduced cells to non-transduced cells, increasing the tissue distribution of this gene, increasing lysosomal gene expression, and the size and number of lysosomes. This, in turn, results in increased clearance of proteins involved in Alzheimer's disease and Parkinson's disease including beta amyloid, tau, and alpha-synuclein. Surprisingly, these changes are evident only 1 week after injection.

In another embodiment, the viral system of the present disclosure serves as the basis of a treatment or prevention of a disease in a subject (animals, including humans). As such, in one embodiment, the present disclosure relates to a method of treating ameliorating or preventing a disorder or disease in a subject comprising administering to the subject a vector carrying a construct that encodes a secretory protein variant of the present disclosure. When the disorder is a neurodegenerative disease, the vectors of the present disclosure may be administered by any suitable administration form, such as being injected into the brain stereotactically, or delivered into CSF of the patient, or delivered intravenously.

In one embodiment, diseases/disorders that can be treated, ameliorated, or prevented with the methods of the present disclosure are associated with dysfunctional lysosomal clearance. The disease/disorder can affect the skeleton, skin, mucous membrane, heart, liver, hematopoetic system, musculature, peripheral nervous system and central nervous system. In one embodiment, the disease or disorder is a neurodegenerative disorder, such as Alzheimer's disease and Parkinson's disease. The constructs of the present disclosure can also be used for other diseases or conditions. For example, when the constructs include HSP70 to aid in cell stress or Cox8, and the mitochondria cytochrome c oxidase subunit 8 (Cox8) to target mitochondria related conditions.

In another embodiment, the diseases/disorders that can be treated, ameliorated, or prevented with the methods of the present disclosure are caused by a pathogen, such as viral infections, bacterial infections and fungal infections.

Advantages of the system of the present disclosure over other strategies in neurodegenerative disease include 1) Amyloid deposition is required to make diagnosis of AD, however, amyloid may not constitute the actual cause of AD. Amyloid could be a side effect of some other metabolic problem, such as a failure of protein clearance. The present disclosure targets an underlying global biochemical deficiency in neurodegenerative disease (the failure of lysosomal clearance) and is not dependent specifically on the particular deposited proteins involved; e.g. it may be effective even if beta-amyloid deposition is not the main problem in Alzheimer's disease; 2) because AAV expresses genes for extended periods, patients would need a single dosing procedure. The techniques described herein could be applicable to many other muscular and neurodegenerative disease paradigms.

In order to aid in the understanding and preparation of the within disclosure, the following illustrative, non-limiting, examples are provided.

### EXAMPLES

### Materials and Methods

DNA Constructs: To generate a TFEB protein that can propagate from a transduced cell to a non-transduced cell, constructs contain a signal sequence for protein secretion, such as the signal sequence derived from the human Amyloid Precursor Protein [MLPGLALLLLAAWTARAL] (SEQ ID NO: 8), and a cell penetrating peptide (the TAT peptide from HIV TVTVVRSVVSVVV (SEQ ID NO: 9)). In addition, the TFEB protein without a cytoskeletal binding site to prevent nuclear transport and without a helix-loop-helix domain to bind to DNA, and lastly a C-terminal Green Fluorescent Protein. The fluorescent protein may be removed to save space in the vector, improve expression, and remove a potential target of the immune system. Initial experiments use constructs that express sTFEB alone, or in with a non-secreted Red Fluorescent Protein (tdTomatoFP). This can be transcribed from its on promoter, or on the same transcript in tandem with sTFEB, with an E2a cleavage site between the 2 proteins, so that the tdTomatoFP and the sTFEB proteins cleaved apart during translation. Currently these constructs are in Adenovirus, and AAV9.

In Vivo Experiments: To demonstrate functionally active propagation of TFEB in a mouse model of AD, experiments are performed in 3×Tg mice (Jackson laboratory; B6; 129-Tg (APPSwe,tauP301L) 1Lfa Psen1^{tm1Mpm}/Mmjax), which displays formation of amyloid plaques and hyperphosphorylated tau. 3×Tg mice develop Aβ deposition as early as 6 months with extensive cortical deposits by 12 months. Hyperphosphorylated tau in the hippocampus is present by 12-15 months. WT mice (Charles River Canada; C57BL/6) will be used as control animals.

Transgenic mice are injected with our adenoviral constructs driving expression of propagating TFEB (WT or mutants) or GFP as a negative control. WT control mice are also injected with adenoviral constructs to assess the effect of TFEB transduction on endogenous Aβ and tau. Stereotaxic surgery is performed to inject animals bilaterally in their hippocampus or lateral ventricles with doses ranging from 10⁷ to 10¹² viral particles per side. Since distribution of viral particles is a major caveat of in vivo studies and thus, two injection sites are chosen to visualize and determine most efficient distribution of our novel propagating TFEB constructs in the brain. In brief, surgery is performed under 2% isoflurane anesthesia and 5 μL of virus is delivered via a 26G Hamilton syringe attached to a microinfusion pump at a rate of 0.5 uL/min over 10 minutes to the lateral ventricles (AP −0.6 mm, ML±1.2 mm, DV −2 mm) or hippocampus (AP −2.4 mm, ML±2 mm, DV −1.5 mm).

To determine if the role of TFEB in lysosomal clearance could display a neuroprotective versus neuro-rescuing effect, animals aged to 3 different timepoints are used: 3 months (pre-phenotypic abnormalities), 5-6 months (onset of abnormalities) and 12 months (post-phenotypic abnormalities). Moreover, to establish a timeline of TFEB effect and efficiency, these aged animals are sacrificed at 3 different end points: 1 week, 1 month and 3 months post-surgery. Thus, to test each dose of TFEB virus, 324 mice are used (6 replicates×3 mice strains×2 injection sites×3 start points×3 end points). All animal care and surgical procedures will be conducted under the guidelines of the Animal Care Committee at Western University (AUP 2016-069).

To visualize distribution of enhanced propagating TFEB, brains are fixed and coronal sections are cut for histological analysis. As our protein of interest (TFEB) is fluorescently-tagged, no additional detection for TFEB is necessary. However, to amplify original TFEB signal, immunostaining for GFP and RFP may be used. Additionally, immunostaining for LAMP1, Aβ₄₀, Aβ₄₂ and phosphorylated-tau are performed using commercially available antibodies.

To assess the ability of propagating TFEB to regulate lysosomal activity, confocal microscopy is performed in order to measure expression, number and size of lysosomes in each TFEB condition (WT versus mutants). Moreover, to study the distribution of TFEB activity in reducing tau and Aβ levels, amyloid plaques or tau neurofibrillary tangles are quantified using NIH ImageJ and Imaris Software. Stereological analysis for neuronal counts are also performed on sections stained with cresyl violet solution. Briefly, unbiased stereology will be performed using the optical dissector method, with data analysis performed using Stereo Investigator Software (MBF Bioscience).

### Results

Microphotographs Showing that sTFEB-GFP Spreads from Cell to Cell in Neuroblastoma Cells

FIG. 2 shows microphotographs of N2A cells transfected with sTFEB-GFP (top), sTFEB-del30-GFP (middle) and sTFEB-DD-GFP (bottom). Cells are immunostained with LAMP1 (magenta). The rightmost panel shows a colocalization channel, comparing the overlap of sTFEB-GFP and lysosomes (LAMP1) by Imaris software (Bitplane). Cells that are transfected with STFEB-GFP also express RFP and are marked by arrowheads. All of the other cells that are green have taken up GFP from the media. Cells that are transfected with sTFEB-GFP show punctate cytoplasmic and nuclear staining, depending on how much sTFEB-GFP is taken up. Some sTFEB-GFP in the cytoplasm colocalizes with lysosomes. Deleting the cytoskeletal binding domain (sTFEB-del30) increases the number of cells with nuclear GFP signal and reduces the amount of STFEB on lysosomes. Deleting both DNA binding and cytoskeletal binding domains (sTFEB-DD) leads to cytoplasmic GFP expression, but also less lysosomal labeling.

STFEB is Delivered from Cell to Cell with High Efficiency

FIG. 3 shows icrophotographs showing that sTFEB (green) but not adenovirus (red) spreads from cell to cell. Cells in the left panel of FIG. 3 are transduced with adenovirus encoding sTFEB-GFP and a non-secreted Red Fluorescent Protein. Media that had been conditioned by these cells, is then added to a second dish of cells (right panels-neuroblastoma cells and cultured human iPSC-derived human neurons.) which then become green (but not red) demonstrating that they have taken up the sTFEB-GFP, but not the virus particles themselves.

sTFEB Increases the Expression of Cathpsin-B, Lysosomes Size and Lysosomal Number

FIG. 4A shows microphotographs of N2a cells (FIG. 4A-top panels) or iPSC-derived neurons (FIG. 4A-bottom panels) which were allowed to take up sTFEB del30 or TFEB/inactive STFEB-DD-GFP for 48 hours from conditioned media. Magic Red (Immunochemistry Technologies) fluorescent substrate for Cathepsin B was added to the media and cells were imaged live.

Lysosome size and number were quantitated using Imaris software. N2A cells which had taken up the constitutively active sTFEB-del30 (compared to control cells) had significantly more lysosomes, larger lysosomes and more CatB enzyme activity (generated by integrating intensity*area) (see FIG. 4B).

Lysosome size and number were quantitated using Imaris software in human iPSC-derived neurons. Neurons which had taken up the constitutively active sTFEB-del30 (compared to control cells) had significantly more lysosomes, larger lysosomes and more CatB enzyme activity (generated by integrating intensity*area) (see FIG. 4C).

sTFEB Increases the Levels of Transcription of Lysosomal Genes and Lysosomal Proteins

With reference to FIG. 5A, N2A cells were treated with constitutively active TFEB-del30 or inactive sTFEB-DD and gene expression of expression was analyzed by rtPCR and normalized to cells exposed to normal media. sTFEB increases Lysosomal gene expression.

With reference to FIG. 5B, N2A cells were treated with sTFEB, constitutively active TFEB-del30 or inactive sTFEB-DD and cells were analyzed by Western Blotting. STFEB increases expression of LAMP1 and CatD proteins but not tubulin (control).

Constitutively Active sTFEB-Del30 Decreases Beta-Amyloid in Cells

N2A neuroblastoma cells were transfected with either the inactive TFEB-DD-GFP (FIG. 6A) or constitutively active sTFEB-del30 (FIG. 6B). After 48 hours, they were loaded overnight with 500 nM of fluorescently labeled beta-amyloid-42 (shown in red). After a further 48 hours of incubation, cells were fixed and imaged. Photomicrographs illustrate that cells that contain inactive TFEB-DD-GFP demonstrated much brighter red signal from beta-amyloid than cells which had taken up constitutively active TEFB-GFP.

Constitutively active sTFEB-del30 reduces neurofibrillary tangles. Human iPSC-derived neurons were transduced with mutant Tau (Tau P301L)-blue fluorescent protein (shown in the photomicrographs in cyan) and treated with tau pre-formed fibrils to generate neurofibrillary tangles. Cells were then exposed to media containing inactive TFEB-DD-GFP (FIG. 7A) or constitutively active sTFEB-del30 (FIG. 7B). Photomicrographs show that cells exposed to inactive TFEB were filled with Tau filaments (FIG. 7A), while cells exposed to active TFEB-del30 have almost no recognizable filaments (FIG. 7B).

Constitutively Active sTFEB Reduces Alpha-Synuclein Deposits.

SHSY5Y neuroblastoma cells (8A and 8B) and human iPSC-derived Neurons (8C and 8D) were transduced with aSyn A53T-BFP (green) and either TFEB-DD-GFP (inactive, 8A and 8C) or sTFEB-del30-GFP (constitutively active, 8B and 8D). After 5 days cells were fixed and imaged by confocal microscopy. The photomicrographs show that cells with which had taken up inactive STFEB-DD-GFP had cytoplasmic synuclein inclusions (arrowheads, 8A and 8C); cells which took up active sTFEB-del30-GFP did not (8B and 8D).

### STFEB-Del30-GFP Greatly Increases TFEB Expression in the Brain Compared to Wild-Type TFEB-GFP, Increases Lysosome Staining and Reduces Alpha-Synuclein

A 14-month-old homozygous M83 mouse received a stereotaxic intraventricular injection of 10\hat{ }7 infectious units of adenovirus overexpressing constitutively active sTFEB-del30-GFP on the left and a conventional wt-TFEB-GFP on the right. After 7 days, mouse brain was perfused, fixed, cryosectioned, and immunostained with an antibody against GFP (green), LAMP1 (red), and aggregated phosphorylated-synuclein, and imaged on a Leica TCS SP8 confocal microscope. The top row of FIG. 9 are photomicrographs showing that TFEB-GFP expression is much higher on the side received the sTFEB construct. The middle row of FIG. 9 shows that the side injected with constitutively active TFEB shows increased LAMP1 staining overall (increased lysosomes), and on high magnification, show increased numbers of lysosomes. The bottom row of FIG. 9 shows tissue stained with an antibody against phosphorylated alpha-synuclein, which is specific to pathological synuclein deposits, and this is reduced on the side injected with sTFEB.

sTFEB-Del30 Clears Beta-Amyloid

A 12-month-old 3×TG Alzheimer's disease mouse was injected into the hippocampus with an adenovirus expressing TFEB-del30-GFP on the left and an adenovirus expressing only a wt-GFP on the right. After 1 week, the mouse was sacrificed and stained for GFP (green) and Beta Amyloid (yellow). The left side injected with sTFEB-del30-GFP shows greatly increased expression and spread compared to the expression of non-spreading GFP (see FIG. 10A).

Amyloid plaques were counted in each hippocampus and the cortex above the hippocampus and the results are shown in the graph of FIG. 10B.

FIGS. 10A and 10B show that there are fewer plaques on the hippocampus and cortex sTFEB-del30 side (left side of 10A, sTFEB) compared to the control side (right side of 10A, GFP).

sTFEB Spreads Beyond its Injection Sites to Clear Beta-Amyloid

A 5 month old 3×TG (APP/PSI/Tau) transgenic mouse was injected on the left, into the hippocampus with an adenovirus expressing sTFEB-GFP and on the right with a the same dose of an adenovirus expressing GFP. The mouse was sacrificed after 2 months, perfused, fixed, cryo-sectioned, and stained with antibodies against GFP and Beta-Amyloid-42 (Aβ42). FIG. 11A shows staining for GFP is at the level of the injection shows that the sTFEB-GFP signal covers the whole hemisphere, and more of the hemisphere than the standard GFP expressing adenovirus. FIG. 11B shows that 3 mm anterior for the injection site, sTFEB-GFP still covers most of the hemisphere, while the regular adenovirus-GFP is seen only in the deeper structures of the brain. sTFEB-GFP injection dramatically decreases the signal of Amyloid-42 immunostaining Aβ at the level of the injection compared the GFP adenovirus (see FIG. 11C). Small rectangles of FIG. 11C are enlarged in FIG. 11D to show that the 7-month AD animal has significant intracellular amyloid and early plaques (on the right), which are both dramatically reduced by sTFEB-GFP (on the left).

### REFERENCES

1. Wolfe D M, Lee J H, Kumar A, Lee S, Orenstein S J, Nixon R A: Autophagy failure in Alzheimer's disease and the role of defective lysosomal acidification. Eur J Neurosci 2013, 37:1949-1961. 23773064
2. Bordi M, Berg M J, Mohan P S, Peterhoff C M, Alldred M J, Che S, Ginsberg S D, Nixon R A: Autophagy flux in C A 1 neurons of Alzheimer hippocampus: Increased induction overburdens failing lysosomes to propel neuritic dystrophy. Autophagy 2016, 12:2467-2483. 27813694
3. Reddy K, Cusack C L, Nnah I C, Khayati K, Saqcena C, Huynh T B, Noggle S A, Ballabio A, Dobrowolski R: Dysregulation of Nutrient Sensing and CLEARance in Presenilin Deficiency. Cell Rep 2016, 14:2166-2179. 26923592
4. Dehay B, Bove J, Rodriguez-Muela N, Perier C, Recasens A, Boya P, Vila M: Pathogenic lysosomal depletion in Parkinson's disease. J Neurosci 2010, 30:12535-12544. 20844148
5. Wallings R L, Humble S W, Ward M E, Wade-Martins R: Lysosomal Dysfunction at the Centre of Parkinson's Disease and Frontotemporal Dementia/Amyotrophic Lateral Sclerosis. Trends Neurosci 2019, 42:899-912. 31704179
6. Cortes C J, La Spada A R: TFEB dysregulation as a driver of autophagy dysfunction in neurodegenerative disease: Molecular mechanisms, cellular processes, and emerging therapeutic opportunities. Neurobiol Dis 2019, 122:83-93. 29852219
7. Palmieri M, Impey S, Kang H, di Ronza A, Pelz C, Sardiello M, Ballabio A: Characterization of the CLEAR network reveals an integrated control of cellular clearance pathways. Hum Mol Genet 2011, 20:3852-3866. 21752829
8. Sardiello M, Palmieri M, di Ronza A, Medina D L, Valenza M, Gennarino V A, Di Malta C, Donaudy F, Embrione V, Polishchuk R S, et al: A gene network regulating lysosomal biogenesis and function. Science 2009, 325:473-477. 19556463
9. Xiao Q, Yan P, Ma X, Liu H, Perez R, Zhu A, Gonzales E, Burchett J M, Schuler D R, Cirrito J R, et al: Enhancing astrocytic lysosome biogenesis facilitates Abeta clearance and attenuates amyloid plaque pathogenesis. J Neurosci 2014, 34:9607-9620. 25031402
10. Xiao Q, Yan P, Ma X, Liu H, Perez R, Zhu A, Gonzales E, Tripoli D L, Czerniewski L, Ballabio A, et al: Neuronal-Targeted TFEB Accelerates Lysosomal Degradation of APP, Reducing Abeta Generation and Amyloid Plaque Pathogenesis. J Neurosci 2015, 35:12137-12151. 26338325
11. Caccamo A, Majumder S, Richardson A, Strong R, Oddo S: Molecular interplay between mammalian target of rapamycin (mTOR), amyloid-beta, and Tau: effects on cognitive impairments. J Biol Chem 2010, 285:13107-13120. 20178983
12. Wang H, Wang R, Carrera I, Xu S, Lakshmana M K: TFEB Overexpression in the P301S Model of Tauopathy Mitigates Increased PHF1 Levels and Lipofuscin Puncta and Rescues Memory Deficits. eNeuro 2016, 3. 27257626
13. Kilpatrick K, Zeng Y, Hancock T, Segatori L: Genetic and chemical activation of TFEB mediates clearance of aggregated alpha-synuclein. PLOS ONE 2015, 10: e0120819. 25790376
14. He X, Yuan W, Li Z, Hou Y, Liu F, Feng J: 6-Hydroxydopamine induces autophagic flux dysfunction by impairing transcription factor EB activation and lysosomal function in dopaminergic neurons and SH-SY5Y cells. Toxicol Lett 2018, 283:58-68. 29170033
15. Decressac M, Mattsson B, Weikop P, Lundblad M, Jakobsson J, Bjorklund A: TFEB-mediated autophagy rescues midbrain dopamine neurons from alpha-synuclein toxicity. Proc Natl Acad Sci USA 2013, 110: E1817-1826. 23610405
16. Arotcarena M L, Bourdenx M, Dutheil N, Thiolat M L, Doudnikoff E, Dovero S, Ballabio A, Fernagut P O, Meissner W G, Bezard E, Dehay B: Transcription factor EB overexpression prevents neurodegeneration in experimental synucleinopathies. JCI Insight 2019, 4. 31434803
17. Mader B J, Pivtoraiko V N, Flippo H M, Klocke B J, Roth K A, Mangieri L R, Shacka J J: Rotenone inhibits autophagic flux prior to inducing cell death. ACS Chem Neurosci 2012, 3:1063-1072. 23259041
18. Wu F, Xu H D, Guan J J, Hou Y S, Gu J H, Zhen X C, Qin Z H: Rotenone impairs autophagic flux and lysosomal functions in Parkinson's disease. Neuroscience 2015, 284:900-911. 25446361
19. Torra A, Parent A, Cuadros T, Rodriguez-Galvan B, Ruiz-Bronchal E, Ballabio A, Bortolozzi A, Vila M, Bove J: Overexpression of TFEB Drives a Pleiotropic Neurotrophic Effect and Prevents Parkinson's Disease-Related Neurodegeneration. Mol Ther 2018, 26:1552-1567. 29628303
20. Ulasov A V, Rosenkranz A A, Sobolev A S: Transcription factors: Time to deliver. J Control Release 2018, 269:24-35. 29113792
21. Snir J A, Suchy M, Lawrence K S, Hudson R H, Pasternak S H, Bartha R: Prolonged In Vivo Retention of a Cathepsin D Targeted Optical Contrast Agent in a Mouse Model of Alzheimer's Disease. J Alzheimers Dis 2015, 48:73-87. 26401930
22. Hudry E, Vandenberghe L H: Therapeutic AAV Gene Transfer to the Nervous System: A Clinical Reality. Neuron 2019, 101:839-862. 30844402
23. Choudhury S R, Hudry E, Maguire C A, Sena-Esteves M, Breakefield X O, Grandi P: Viral vectors for therapy of neurologic diseases. Neuropharmacology 2017, 120:63-80. 26905292
24. Wang Y, Wang F, Wang R, Zhao P, Xia Q: 2A self-cleaving peptide-based multi-gene expression system in the silkworm Bombyx mori. Sci Rep 2015, 5:16273. 26537835
25. Roczniak-Ferguson A, Petit C S, Froehlich F, Qian S, Ky J, Angarola B, Walther T C, Ferguson S M: The transcription factor TFEB links mTORC1 signaling to transcriptional control of lysosome homeostasis. Sci Signal 2012, 5: ra42. 22692423
26. Fisher D E, Carr C S, Parent L A, Sharp P A: TFEB has DNA-binding and oligomerization properties of a unique helix-loop-helix/leucine-zipper family. Genes Dev 1991, 5:2342-2352. 1748288
27. Griffin J M, Fackelmeier B, Fong D M, Mouravlev A, Young D, O'Carroll S J: Astrocyte-selective AAV gene therapy through the endogenous GFAP promoter results in robust transduction in the rat spinal cord following injury. Gene Ther 2019, 26:198-210. 30962538

Through the embodiments that are illustrated and described, the currently contemplated best mode of making and using the disclosure is described. Without further elaboration, it is believed that one of ordinary skill in the art can, based on the description presented herein, utilize the present disclosure to the full extent. All publications cited herein are incorporated by reference.

Although the description above contains many specificities, these should not be construed as limiting the scope of the disclosure, but as merely providing illustrations of some of the presently embodiments of this disclosure.

Sequence Listing

SEQ ID NO: 1 TFEB (wild type protein)
Met Ala Ser Arg Ile Gly Leu Arg Met Gln Leu Met Arg Glu Gln Ala Gln Gln Glu Glu Gln Arg
Glu Arg Met Gln Gln Gln Ala Val Met His Tyr Met Gln Gln Gln Gln Gln Gln Gln Gln Gln Gln
Leu Gly Gly Pro Pro Thr Pro Ala Ile Asn Thr Pro Val His Phe Gln Ser Pro Pro Pro Val Pro Gly
Glu Val Leu Lys Val Gln Ser Tyr Leu Glu Asn Pro Thr Ser Tyr His Leu Gln Gln Ser Gln His Gln
Lys Val Arg Glu Tyr Leu Ser Glu Thr Tyr Gly Asn Lys Phe Ala Ala His Ile Ser Pro Ala Gln Gly
Ser Pro Lys Pro Pro Pro Ala Ala Ser Pro Gly Val Arg Ala Gly His Val Leu Ser Ser Ser Ala Gly
Asn Ser Ala Pro Asn Ser Pro Met Ala Met Leu His Ile Gly Ser Asn Pro Glu Arg Glu Leu Asp Asp
Val Ile Asp Asn Ile Met Arg Leu Asp Asp Val Leu Gly Tyr Ile Asn Pro Glu Met Gln Met Pro Asn
Thr Leu Pro Leu Ser Ser Ser His Leu Asn Val Tyr Ser Ser Asp Pro Gln Val Thr Ala Ser Leu Val
Gly Val Thr Ser Ser Ser Cys Pro Ala Asp Leu Thr Gln Lys Arg Glu Leu Thr Asp Ala Glu Ser Arg
Ala Leu Ala Lys Glu Arg Gln Lys Lys Asp Asn His Asn Leu Ile Glu Arg Arg Arg Arg Phe Asn
Ile Asn Asp Arg Ile Lys Glu Leu Gly Met Leu Ile Pro Lys Ala Asn Asp Leu Asp Val Arg Trp Asn
Lys Gly Thr Ile Leu Lys Ala Ser Val Asp Tyr Ile Arg Arg Met Gln Lys Asp Leu Gln Lys Ser Arg
Glu Leu Glu Asn His Ser Arg Arg Leu Glu Met Thr Asn Lys Gln Leu Trp Leu Arg Ile Gln Glu
Leu Glu Met Gln Ala Arg Val His Gly Leu Pro Thr Thr Ser Pro Ser Gly Met Asn Met Ala Glu
Leu Ala Gln Gln Val Val Lys Gln Glu Leu Pro Ser Glu Glu Gly Pro Gly Glu Ala Leu Met Leu
Gly Ala Glu Val Pro Asp Pro Glu Pro Leu Pro Ala Leu Pro Pro Gln Ala Pro Leu Pro Leu Pro Thr
Gln Pro Pro Ser Pro Phe His His Leu Asp Phe Ser His Ser Leu Ser Phe Gly Gly Arg Glu Asp Glu
Gly Pro Pro Gly Tyr Pro Glu Pro Leu Ala Pro Gly His Gly Ser Pro Phe Pro Ser Leu Ser Lys Lys
Asp Leu Asp Leu Met Leu Leu Asp Asp Ser Leu Leu Pro Leu Ala Ser Asp Pro Leu Leu Ser Thr
Met Ser Pro Glu Ala Ser Lys Ala Ser Ser Arg Arg Ser Ser Phe Ser Met Glu Glu Gly Asp Val Leu
Lys Val Pro Arg Ala Arg Asp Pro Pro Val Ala Thr
SEQ ID NO: 2 (protein) sTFEB SS-TAT-TFEB
Met Leu Pro Gly Leu Ala Leu Leu Leu Leu Ala Ala Trp Thr Ala Arg Ala Leu Asn Gly Tyr Gly
Arg Lys Lys Arg Arg Gln Arg Arg Arg Gly Met Ala Ser Arg Ile Gly Leu Arg Met Gln Leu Met
Arg Glu Gln Ala Gln Gln Glu Glu Gln Arg Glu Arg Met Gln Gln Gln Ala Val Met His Tyr Met
Gln Gln Gln Gln Gln Gln Gln Gln Gln Gln Leu Gly Gly Pro Pro Thr Pro Ala Ile Asn Thr Pro Val
His Phe Gln Ser Pro Pro Pro Val Pro Gly Glu Val Leu Lys Val Gln Ser Tyr Leu Glu Asn Pro Thr
Ser Tyr His Leu Gln Gln Ser Gln His Gln Lys Val Arg Glu Tyr Leu Ser Glu Thr Tyr Gly Asn Lys
Phe Ala Ala His Ile Ser Pro Ala Gln Gly Ser Pro Lys Pro Pro Pro Ala Ala Ser Pro Gly Val Arg
Ala Gly His Val Leu Ser Ser Ser Ala Gly Asn Ser Ala Pro Asn Ser Pro Met Ala Met Leu His Ile
Gly Ser Asn Pro Glu Arg Glu Leu Asp Asp Val Ile Asp Asn Ile Met Arg Leu Asp Asp Val Leu
Gly Tyr Ile Asn Pro Glu Met Gln Met Pro Asn Thr Leu Pro Leu Ser Ser Ser His Leu Asn Val Tyr
Ser Ser Asp Pro Gln Val Thr Ala Ser Leu Val Gly Val Thr Ser Ser Ser Cys Pro Ala Asp Leu Thr
Gln Lys Arg Glu Leu Thr Asp Ala Glu Ser Arg Ala Leu Ala Lys Glu Arg Gln Lys Lys Asp Asn
His Asn Leu Ile Glu Arg Arg Arg Arg Phe Asn Ile Asn Asp Arg Ile Lys Glu Leu Gly Met Leu Ile
Pro Lys Ala Asn Asp Leu Asp Val Arg Trp Asn Lys Gly Thr Ile Leu Lys Ala Ser Val Asp Tyr Ile
Arg Arg Met Gln Lys Asp Leu Gln Lys Ser Arg Glu Leu Glu Asn His Ser Arg Arg Leu Glu Met
Thr Asn Lys Gln Leu Trp Leu Arg Ile Gln Glu Leu Glu Met Gln Ala Arg Val His Gly Leu Pro
Thr Thr Ser Pro Ser Gly Met Asn Met Ala Glu Leu Ala Gln Gln Val Val Lys Gln Glu Leu Pro Ser
Glu Glu Gly Pro Gly Glu Ala Leu Met Leu Gly Ala Glu Val Pro Asp Pro Glu Pro Leu Pro Ala
Leu Pro Pro Gln Ala Pro Leu Pro Leu Pro Thr Gln Pro Pro Ser Pro Phe His His Leu Asp Phe Ser
His Ser Leu Ser Phe Gly Gly Arg Glu Asp Glu Gly Pro Pro Gly Tyr Pro Glu Pro Leu Ala Pro Gly
His Gly Ser Pro Phe Pro Ser Leu Ser Lys Lys Asp Leu Asp Leu Met Leu Leu Asp Asp Ser Leu
Leu Pro Leu Ala Ser Asp Pro Leu Leu Ser Thr Met Ser Pro Glu Ala Ser Lys Ala Ser Ser Arg Arg
Ser Ser Phe Ser Met Glu Glu Gly Asp Val Leu Lys Val Pro Arg Ala Arg Asp Pro Pro Val Ala Thr
SEQ ID NO: 3 (protein) sTFEB-del30 (SS-TAT-TFEB-del 30)
Met Leu Pro Gly Leu Ala Leu Leu Leu Leu Ala Ala Trp Thr Ala Arg Ala Leu Asn Gly Tyr Gly
Arg Lys Lys Arg Arg Gln Arg Arg Arg Gly Met His Tyr Met Gln Gln Gln Gln Gln Gln Gln Gln
Gln Gln Leu Gly Gly Pro Pro Thr Pro Ala Ile Asn Thr Pro Val His Phe Gln Ser Pro Pro Pro Val
Pro Gly Glu Val Leu Lys Val Gln Ser Tyr Leu Glu Asn Pro Thr Ser Tyr His Leu Gln Gln Ser Gln
His Gln Lys Val Arg Glu Tyr Leu Ser Glu Thr Tyr Gly Asn Lys Phe Ala Ala His Ile Ser Pro Ala
Gln Gly Ser Pro Lys Pro Pro Pro Ala Ala Ser Pro Gly Val Arg Ala Gly His Val Leu Ser Ser Ser
Ala Gly Asn Ser Ala Pro Asn Ser Pro Met Ala Met Leu His Ile Gly Ser Asn Pro Glu Arg Glu Leu
Asp Asp Val Ile Asp Asn Ile Met Arg Leu Asp Asp Val Leu Gly Tyr Ile Asn Pro Glu Met Gln
Met Pro Asn Thr Leu Pro Leu Ser Ser Ser His Leu Asn Val Tyr Ser Ser Asp Pro Gln Val Thr Ala
Ser Leu Val Gly Val Thr Ser Ser Ser Cys Pro Ala Asp Leu Thr Gln Lys Arg Glu Leu Thr Asp Ala
Glu Ser Arg Ala Leu Ala Lys Glu Arg Gln Lys Lys Asp Asn His Asn Leu Ile Glu Arg Arg Arg
Arg Phe Asn Ile Asn Asp Arg Ile Lys Glu Leu Gly Met Leu Ile Pro Lys Ala Asn Asp Leu Asp Val
Arg Trp Asn Lys Gly Thr Ile Leu Lys Ala Ser Val Asp Tyr Ile Arg Arg Met Gln Lys Asp Leu Gln
Lys Ser Arg Glu Leu Glu Asn His Ser Arg Arg Leu Glu Met Thr Asn Lys Gln Leu Trp Leu Arg
Ile Gln Glu Leu Glu Met Gln Ala Arg Val His Gly Leu Pro Thr Thr Ser Pro Ser Gly Met Asn Met
Ala Glu Leu Ala Gln Gln Val Val Lys Gln Glu Leu Pro Ser Glu Glu Gly Pro Gly Glu Ala Leu Met
Leu Gly Ala Glu Val Pro Asp Pro Glu Pro Leu Pro Ala Leu Pro Pro Gln Ala Pro Leu Pro Leu Pro
Thr Gln Pro Pro Ser Pro Phe His His Leu Asp Phe Ser His Ser Leu Ser Phe Gly Gly Arg Glu Asp
Glu Gly Pro Pro Gly Tyr Pro Glu Pro Leu Ala Pro Gly His Gly Ser Pro Phe Pro Ser Leu Ser Lys
Lys Asp Leu Asp Leu Met Leu Leu Asp Asp Ser Leu Leu Pro Leu Ala Ser Asp Pro Leu Leu Ser
Thr Met Ser Pro Glu Ala Ser Lys Ala Ser Ser Arg Arg Ser Ser Phe Ser Met Glu Glu Gly Asp Val
Leu Lys Val Pro Arg Ala Arg Asp Pro Pro Val Ala Thr
SEQ ID NO: 4 (Protein) sTFEB-DD (SS-TAT-TFEB-DD)
Met Leu Pro Gly Leu Ala Leu Leu Leu Leu Ala Ala Trp Thr Ala Arg Ala Leu Asn Gly Tyr Gly
Arg Lys Lys Arg Arg Gln Arg Arg Arg Gly Met His Tyr Met Gln Gln Gln Gln Gln Gln Gln Gln
Gln Gln Leu Gly Gly Pro Pro Thr Pro Ala Ile Asn Thr Pro Val His Phe Gln Ser Pro Pro Pro Val
Pro Gly Glu Val Leu Lys Val Gln Ser Tyr Leu Glu Asn Pro Thr Ser Tyr His Leu Gln Gln Ser Gln
His Gln Lys Val Arg Glu Tyr Leu Ser Glu Thr Tyr Gly Asn Lys Phe Ala Ala His Ile Ser Pro Ala
Gln Gly Ser Pro Lys Pro Pro Pro Ala Ala Ser Pro Gly Val Arg Ala Gly His Val Leu Ser Ser Ser
Ala Gly Asn Ser Ala Pro Asn Ser Pro Met Ala Met Leu His Ile Gly Ser Asn Pro Glu Arg Glu Leu
Asp Asp Val Ile Asp Asn Ile Met Arg Leu Asp Asp Val Leu Gly Tyr Ile Asn Pro Glu Met Gln
Met Pro Asn Thr Leu Pro Leu Ser Ser Ser His Leu Asn Val Tyr Ser Ser Asp Pro Gln Val Thr Ala
Ser Leu Val Gly Val Thr Ser Ser Ser Cys Pro Ala Asp Leu Thr Gln Lys Arg Glu Leu Thr Asp Ala
Glu Ser Arg Ala Asp Arg Ile Lys Glu Leu Gly Met Leu Ile Pro Lys Ala Asn Asp Leu Asp Val Arg
Trp Asn Lys Gly Thr Ile Leu Lys Ala Ser Val Asp Tyr Ile Arg Arg Met Gln Lys Asp Leu Gln Lys
Ser Arg Glu Leu Glu Asn His Ser Arg Arg Leu Glu Met Thr Asn Lys Gln Leu Trp Leu Arg Ile
Gln Glu Leu Glu Met Gln Ala Arg Val His Gly Leu Pro Thr Thr Ser Pro Ser Gly Met Asn Met
Ala Glu Leu Ala Gln Gln Val Val Lys Gln Glu Leu Pro Ser Glu Glu Gly Pro Gly Glu Ala Leu Met
Leu Gly Ala Glu Val Pro Asp Pro Glu Pro Leu Pro Ala Leu Pro Pro Gln Ala Pro Leu Pro Leu Pro
Thr Gln Pro Pro Ser Pro Phe His His Leu Asp Phe Ser His Ser Leu Ser Phe Gly Gly Arg Glu Asp
Glu Gly Pro Pro Gly Tyr Pro Glu Pro Leu Ala Pro Gly His Gly Ser Pro Phe Pro Ser Leu Ser Lys
Lys Asp Leu Asp Leu Met Leu Leu Asp Asp Ser Leu Leu Pro Leu Ala Ser Asp Pro Leu Leu Ser
Thr Met Ser Pro Glu Ala Ser Lys Ala Ser Ser Arg Arg Ser Ser Phe Ser Met Glu Glu Gly Asp Val
Leu Lys Val Pro Arg Ala Arg Asp Pro Pro Val Ala Thr
SEQ ID NO: 5 DNA STFEB (SS TAT_TFEB)
atgctgcccggtttggcactgctcctgctggccgcctggacggctcgggcgctgaacggttacggtcgtaagaagcgtcgtcagcgtcgtc
gtggcatggcgtcacgcatagggttgcgcatgcagctcatgcgggagcaggcgcagcaggaggagcaacgggagcgcatgcagcaac
aggctgtcatgcattacatgcagcagcagcagcagcagcaacagcagcagctcggagggccgcccaccccggccatcaatacccccgt
ccacttccagtcgccaccacctgtgcctggggaggtgttgaaggtgcagtcctacctggagaatcccacatcctaccatctgcagcagtcgc
agcatcagaaggtgcgggagtacctgtccgagacctatgggaacaagtttgctgcccacatcagcccagcccagggctctccgaaacccc
caccagccgcctccccaggggtgcgagctggacacgtgctgtcctcctccgctggcaacagtgctcccaatagccccatggccatgctgc
acattggctccaaccctgagagggagttggatgatgtcattgacaacattatgcgtctggacgatgtccttggctacatcaatcctgaaatgca
gatgcccaacacgctacccctgtccagcagccacctgaatgtgtacagcagcgacccccaggtcacagcctccctggtgggcgtcacca
gcagctcctgccctgcggacctgacccagaagcgagagctcacagatgctgagagcagggccctggccaaggagcggcagaagaaag
acaatcacaacttaattgaaaggagacgaaggttcaacatcaatgaccgcatcaaggagttgggaatgctgatccccaaggccaatgacct
ggacgtgcgctggaacaagggcaccatcctcaaggcctctgtggattacatccggaggatgcagaaggacctgcaaaagtccagggagc
tggagaaccactctcgccgcctggagatgaccaacaagcagctctggctccgtatccaggagctggagatgcaggctcgagtgcacggc
ctccctaccacctccccgtccggcatgaacatggctgagctggcccagcaggtggtgaagcaggagctgcctagcgaagagggcccag
gggaggccctgatgctgggggctgaggtccctgaccctgagccactgccagctctgcccccgcaagccccgctgcccctgcccaccca
gccaccgtccccattccatcacctggacttcagccacagcctgagctttgggggcagggaggacgagggtcccccgggctaccccgaac
ccctggcgccggggcatggctccccattccccagcctgtccaagaaggatctggacctcatgctcctggacgactcactgctaccgctggc
ctctgatccacttctgtccaccatgtcccccgaggcctccaaggccagcagccgccggagcagcttcagcatggaggagggcgatgtgct
gaaggtaccgcgggcccgggatccaccggtcgccacctaa
SEQ ID NO: 6 DNA STFEB-del30 (SS-TAT-TFEB-del30)
atgctgcccggtttggcactgctcctgctggccgcctggacggctcgggcgctgaacggttacggtcgtaagaagcgtcgtcagcgtcgtc
gtggcatgcattacatgcagcagcagcagcagcagcaacagcagcagctcggagggccgcccaccccggccatcaatacccccgtcca
cttccagtcgccaccacctgtgcctggggaggtgttgaaggtgcagtcctacctggagaatcccacatcctaccatctgcagcagtogcag
catcagaaggtgcgggagtacctgtccgagacctatgggaacaagtttgctgcccacatcagcccagcccagggctctccgaaaccccca
ccagccgcctccccaggggtgcgagctggacacgtgctgtcctcctccgctggcaacagtgctcccaatagccccatggccatgctgcac
attggctccaaccctgagagggagttggatgatgtcattgacaacattatgcgtctggacgatgtccttggctacatcaatcctgaaatgcaga
tgcccaacacgctacccctgtccagcagccacctgaatgtgtacagcagcgacccccaggtcacagcctccctggtgggcgtcaccagca
gctcctgccctgcggacctgacccagaagcgagagctcacagatgctgagagcagggccctggccaaggagcggcagaagaaagaca
atcacaacttaattgaaaggagacgaaggttcaacatcaatgaccgcatcaaggagttgggaatgctgatccccaaggccaatgacctgga
cgtgcgctggaacaagggcaccatcctcaaggcctctgtggattacatccggaggatgcagaaggacctgcaaaagtccagggagctgg
agaaccactctcgccgcctggagatgaccaacaagcagctctggctccgtatccaggagctggagatgcaggctcgagtgcacggcctc
cctaccacctccccgtccggcatgaacatggctgagctggcccagcaggtggtgaagcaggagctgcctagcgaagagggcccagggg
aggccctgatgctgggggctgaggtccctgaccctgagccactgccagctctgcccccgcaagccccgctgcccctgcccacccagcca
ccgtccccattccatcacctggacttcagccacagcctgagctttgggggcagggaggacgagggtcccccgggctaccccgaacccct
ggcgccggggcatggctccccattccccagcctgtccaagaaggatctggacctcatgctcctggacgactcactgctaccgctggcctct
gatccacttctgtccaccatgtcccccgaggcctccaaggccagcagccgccggagcagcttcagcatggaggagggcgatgtgctgaa
ggtaccgcgggcccgggatccaccggtcgccacctaa
SEQ ID NO: 7 DNA STFEB-DD (SS-TAT-TFEB-DD)
atgctgcccggtttggcactgctcctgctggccgcctggacggctcgggcgctgaacggttacggtcgtaagaagcgtcgtcagcgtcgtc
gtggcatgcattacatgcagcagcagcagcagcagcaacagcagcagctcggagggccgcccaccccggccatcaatacccccgtcca
cttccagtcgccaccacctgtgcctggggaggtgttgaaggtgcagtcctacctggagaatcccacatcctaccatctgcagcagtcgcag
catcagaaggtgcgggagtacctgtccgagacctatgggaacaagtttgctgcccacatcagcccagcccagggctctccgaaaccccca
ccagccgcctccccaggggtgcgagctggacacgtgctgtcctcctccgctggcaacagtgctcccaatagccccatggccatgctgcac
attggctccaaccctgagagggagttggatgatgtcattgacaacattatgcgtctggacgatgtccttggctacatcaatcctgaaatgcaga
tgcccaacacgctacccctgtccagcagccacctgaatgtgtacagcagcgacccccaggtcacagcctccctggtgggcgtcaccagca
gctcctgccctgcggacctgacccagaagcgagagctcacagatgctgagagcagggccgaccgcatcaaggagttgggaatgctgatc
cccaaggccaatgacctggacgtgcgctggaacaagggcaccatcctcaaggcctctgtggattacatccggaggatgcagaaggacct
gcaaaagtccagggagctggagaaccactctcgccgcctggagatgaccaacaagcagctctggctccgtatccaggagctggagatgc
aggctcgagtgcacggcctccctaccacctccccgtccggcatgaacatggctgagctggcccagcaggtggtgaagcaggagctgcct
agcgaagagggcccaggggaggccctgatgctgggggctgaggtccctgaccctgagccactgccagctctgcccccgcaagccccg
ctgcccctgcccacccagccaccgtccccattccatcacctggacttcagccacagcctgagctttgggggcagggaggacgagggtccc
ccgggctaccccgaacccctggcgccggggcatggctccccattccccagcctgtccaagaaggatctggacctcatgctcctggacgac
tcactgctaccgctggcctctgatccacttctgtccaccatgtcccccgaggcctccaaggccagcagccgccggagcagcttcagcatgg
aggagggcgatgtgctgaaggtaccgcgggcccgggatccaccggtcgccacctaa

1. A nucleic acid construct comprising a polynucleotide that encodes a secretory protein variant, the secretory protein variant comprising a non-secretory protein having a signal sequence domain and a cell penetrating peptide (CPP) sequence domain.

2. (canceled)

3. The nucleic acid construct of claim 1, wherein the non-secretory protein is a transcription factor.

4. The nucleic acid construct of claim 1, wherein the secretory protein variant is a secreted TFEB (sTFEB) comprising a wild-type TFEB having the signal sequence and the self-penetrating sequence.

5. The nucleic acid construct of claim 1, wherein the non-secretory protein is a HSP or Cox8.

6. The nucleic acid construct of claim 1, wherein the polynucleotide is devoid of a sequence that encodes a cytoskeletal binding site and/or devoid a sequence that encodes a DNA binding domain.

7. (canceled)

8. The nucleic acid construct of claim 1, wherein the self-penetrating sequence includes a TAT sequence, Pep-1, PVEC, Polyarginine (R₉, R₈), Penetratin, PVEC, MPG, R₆/W₃, SAP, CyLoP-1, gH 625, GALA, TP10, CADY, L17E, MPPs, Ac-1, Ent ac-1, Ac-2, Peptide 3, RR5-App, RR4-App, RR3-aPP, TATp-D, R4-R4, R5, R5, [WR]4, Cyclic Tat, cFΦR4, Danamide D, Pro-(Xaa)4-Tyr, Cyclic sC18, RRRRΦF, BIM SAHB9 SAH-SOS1, 4-R, 4-W, Sp-CC-PEG₂₀₀₀, K₁₀(QW)₆, YTA4, v2 or W₃.

9. The nucleic acid construct of claim 1, wherein the signal sequence domain is derived from of the human Amyloid Precursor protein

10. The nucleic acid construct of claim 1, wherein the nucleic acid construct comprises an inducible promoter or a constitutive promoter operatively linked to the polynucleotide that encodes the non-secretory protein.

11-12. (canceled)

13. The nucleic acid construct of claim 1, wherein the polynucleotide comprises SEQ ID NO: 5, SEQ ID NO: 6 or SEQ ID NO: 7.

14-15. (canceled)

16. A recombinant vector comprising the nucleic acid construct of claim 1.

17. The recombinant vector of claim 16, wherein the recombinant vector is a viral vector.

18. (canceled)

19. The recombinant vector of claim 17, wherein the viral vector is an adeno associated virus (AAV).

20-28. (canceled)

29. The nucleic acid construct of claim 1, wherein the secretory protein variant comprises SEQ ID NO: 2, SEQ ID NO: 3 or SEQ ID NO: 4.

30-31. (canceled)

32. A method of treating, ameliorating or preventing a disease in a subject, the method comprising administering to the subject a vector, wherein the vector comprises a polynucleotide construct that encodes a recombinant secretory protein variant comprising a non-secretory protein having a signal sequence domain and a cell penetrating peptide (CPP) domain.

33. The method of claim 32, wherein the vector is a viral vector.

34-37. (canceled)

38. The method according to claim 32, wherein the non-secretory protein is a Transcription Factor EB (TFEB), and wherein the recombinant secretory protein variant comprises a wild-type TFEB having the signal sequence domain and the cell-penetrating peptide domain.

39. The method of claim 32, wherein the non-secretory protein is a HSP or Cox8.

40. The method of claim 32, wherein the CPP domain includes a TAT sequence, Pep-1, PVEC, Polyarginine (R9, R8), Penetratin, PVEC, MPG, R6/W3, SAP, CyLoP-1, gH 625, GALA, TP10, CADY, L17E, MPPs, Ac-1, Ent ac-1, Ac-2, Peptide 3, RR5-App, RR4-App, RR3-aPP, TATp-D, R4-R4, R5, R5, [WR]4, Cyclic Tat, cFΦR4, Danamide D, Pro-(Xaa)4-Tyr, Cyclic sC18, RRRRΦF, BIM SAHB9 SAH-SOS1, 4-R, 4-W, Sp-CC-PEG2000, K10(QW)6, YTA4, v2 or W3.

41. The method of claim 32, wherein the signal sequence domain includes a signal peptide of the human Amyloid Precursor protein.

42. The method of claim 32, wherein the recombinant secretory protein variant is devoid of a cytoskeletal binding site (STFEB del30).

43. The method of claim 32, wherein the recombinant secretory protein comprises SEQ ID NO: 2, SEQ ID NO: 3 or SEQ ID NO: 4.

44-45. (canceled)

46. The method of claim 32, wherein the disease is associated with dysfunctional lysosomal clearance.

47. The method of claim 32, wherein the disease affects the skeleton, skin, mucous membrane, heart, liver, kidney, hematopoietic system, musculature, peripheral nervous system and central nervous system.

48. The method of claim 47, wherein the disease is a neurodegenerative disease.

49. The method of claim 48, wherein the neurodegenerative disease is Alzheimer's disease or Parkinson's disease.

50. The method of claim 32, wherein the disease is caused by a pathogen.

51. (canceled)

52. The method of claim 32, wherein the disease is muscular dystrophy or MS.

53-59. (canceled)
